SEQUENCE LISTING (13 sequences)

SEQ ID NO: 1 - DNA, 21 bp, Artificial Sequence
  source: /note=“Description of Artificial Sequence Synthetic oligonucleotide”
ccacgtcactcacaagtttct

SEQ ID NO: 2 - DNA, 21 bp, Artificial Sequence
  source: /note=“Description of Artificial Sequence Synthetic oligonucleotide”
ggtttggagtctggactaagc

SEQ ID NO: 3 - DNA, 21 bp, Artificial Sequence
  source: /note=“Description of Artificial Sequence Synthetic oligonucleotide”
ggaagtcaccagaggacaaga

SEQ ID NO: 4 - DNA, 21 bp, Artificial Sequence
  source: /note=“Description of Artificial Sequence Synthetic oligonucleotide”
aacttacgctgagtacttcga

SEQ ID NO: 5 - DNA, 21 bp, Artificial Sequence
  source: /note=“Description of Artificial Sequence Synthetic oligonucleotide”
gcatcatctttgagtactacg

SEQ ID NO: 6 - DNA, 21 bp, Artificial Sequence
  source: /note=“Description of Artificial Sequence Synthetic oligonucleotide”
cttctcggatgatcagaaaga

SEQ ID NO: 7 - DNA, 21 bp, Artificial Sequence
  source: /note=“Description of Artificial Sequence Synthetic oligonucleotide”
gaatatgtggaatagtgtaaa

SEQ ID NO: 8 - DNA, 21 bp, Artificial Sequence
  source: /note=“Description of Artificial Sequence Synthetic oligonucleotide”
gaatagtgtaaatatgacaga

SEQ ID NO: 9 - DNA, 21 bp, Artificial Sequence
  source: /note=“Description of Artificial Sequence Synthetic oligonucleotide”
acatgtatatactactattta

SEQ ID NO: 10 - protein, 9 aa, Unknown
  source: /note=“Description of Unknown wild type human, mouse or rat B56beta (PPP2r5b) sequence”
RSHSSSQFR

SEQ ID NO: 11 - protein, 26 aa, Unknown
  source: /note=“Description of Unknown wild type human, mouse or rat B56beta sequence”
GFSRRSLRRARPRRSHSSSQFRYQSN

SEQ ID NO: 12 - protein, 26 aa, Artificial Sequence
  source: /note=“Description of Artificial Sequence Synthetic peptide”
GFARRALRRARPRRAHAAAQFRYQSN

SEQ ID NO: 13 - protein, 11 aa, Unknown
  source: /note=“Description of Unknown CLK family LAMMER kinase peptide”
EHLAMMERILG

(19) United States
(12) Patent Application Publication
(10) Pub. No.: US 2018/0296537 A1
(43) Pub. Date: Oct. 18, 2018

(54) METHODS AND COMPOSITIONS FOR DIAGNOSING, TREATING, AND MONITORING TREATMENT OF SHANK3 DEFICIENCY ASSOCIATED DISORDERS

(71) Applicant: Novartis AG, Basel (CH)

(72) Inventors: Michael BIDINOSTI, Basel (CH); Ivan GALIMBERTI, Basel (CH)

(21) Appl. No.: 15/578,832

(22) PCT Filed: Jun. 3, 2016
(86) PCT No.: PCT/IB2016/053278

§ 371 Date: Dec. 1, 2017

Related U.S. Application Data
(60) Provisional application No. 62/171,264, filed on Jun. 5, 2015.

(51) Int. Cl. A61K 31/428; A61K 31/353; A61P 25/28; A61P 25/00; A61K 38/30; C12Q 1/48

(52) U.S. Cl. CPC A61K 31/428; A61K 31/353; A61P 25/28; A61P 25/00; G01N 2800/302; C12Q 1/485; G01N 2333/912; G01N 2800/28; A61K 38/30

(57) ABSTRACT

The invention provides novel methods and compositions for diagnosing, treating, and monitoring treatment of Shank3 (SH3 and multiple ankyrin repeat domains 3) deficiency associated disorders.

### TECHNICAL FIELD

The present invention provides methods and compositions for diagnosing, treating, and monitoring treatment of Shank3 (SH3 and multiple ankyrin repeat domains 3) deficiency associated disorders.

### BACKGROUND

SH3 and multiple ankyrin repeat domains 3 (Shank3, also known as PSAP2, PROSAP2 (proline-rich synapse-associated protein 2), SPANK-2, SCZD15, DEL22q13.3, or KIAA1650) is a large scaffolding protein that regulates the structural organization of dendritic spines. In human, Shank3 protein is encoded by the SHANK3 gene on chromosome 22. Shank3 primarily functions to assemble and maintain excitatory postsynaptic densities (PSD) by bridging structural proteins, signaling and cytoskeletal molecules, and glutamate receptors (Jiang and Ehlers, 2013, Neuron 78: 8-27). PSDs are most commonly found on dendritic spines of pyramidal neurons of the neocortex and hippocampus and Purkinje cells of the cerebellum, as well as on dendritic shafts at sites of contact with interneurons in the neocortex and hippocampus, as well as motoneurons in the spinal cord. As such the PSD represents a critical organelle for glutamatergic transmission. It has been shown that the SHANK proteins (including SHANK3) constitute a major part of the PSD, representing about 5% of the total protein molecules and total protein mass in the postsynaptic site (Sugiyama et al., 2005, Nature Methods 2 (9): 677-84). As it has been postulated that SHANK proteins may nucleate the protein framework for the PSD, a recent study examined the ability of the sterile alpha motif (SAM) of SHANK3 to form polymers by self-association and found that the SAM domain of SHANK3 was able to self-associate, giving rise to large sheets of parallel fibers (Baron et al., 2006, Science 311 (5760): 531-5). These studies support the hypothesis that sheets of the SHANK proteins can form the scaffold or platform onto which the PSD is constructed. With the SHANK proteins (including SHANK3) forming a molecular platform onto which the PSD protein complex can be constructed, other proteins and protein complexes of the PSD can associate with the SHANK platform. Of the various protein complexes associated with glutamatergic synapses, there is good evidence that the NMDA receptor complex (NRC), the metabotropic glutamate receptor complex (mGC), and the AMPA receptor complex (ARC) associate with the SHANK platform (see Boeckers, 2006, Cell and Tissue Research 326 (2): 409-22).

Accordingly, Shank3 plays a critical role in dendritic spine formation. Reduction of Shank3 expression results in loss of dendritic spine density in most model systems (Peca et al., 2011, Nature 472: 437-442; Roussignol et al., 2005. The Journal of Neuroscience 25: 3560-3570; Verpelli et al., 2011, The Journal of Biological Chemistry 286: 34839-34850; Wang et al., 2011, Human Molecular Genetics 20: 3093-3108). Conversely, overexpression of Shank3 is sufficient for spine formation in apsiny neurons (Roussignol et al., 2005. The Journal of Neuroscience 25: 3560-3570) or enhancement of spine number in mice (Han et al., 2013, Nature 503: 72-77). This is consistent for a role of Shank 3 in the regulation of actin polymerization (Duffney et al., 2013, The Journal of Neuroscience 33: 15767-15778; Durand, et al., 2012, Molecular Psychiatry 17: 71-84; Han et al., Nature 503: 72-77). To date, little is known about the impact of Shank3 deficiency on the activity of canonical signaling pathways.

### SUMMARY OF THE INVENTION

Provided herein are methods and compositions for diagnosing, treating, and monitoring treatment of Shank3 deficiency associated disorders, e.g., Phelan-McDermid syndrome, autism spectrum disorder, intellectual disability, or schizophrenia. The present invention is based, at least in part, on the discovery that Shank3 deficiency leads to impaired degradation of CLK2 (cdc2-like kinase 2) protein, which phosphorylates the protein phosphatase 2A (PP2A) regulatory subunit B56β and results in recruitment of the PP2A catalytic subunit to protein kinase B (PKB or Akt) and dephosphorylation of Akt. The present invention shows that restoration of Akt activation, either directly or via CLK2 or PP2A inhibition, can rescue the reduced dendritic spine density and impaired frequency of synaptic transmission in Shank3-deficient neurons. Accordingly, provided herein are methods of treating Shank3 deficiency, e.g., Phelan-McDermid syndrome, autism spectrum disorder, intellectual disability, or schizophrenia, in a subject in need of treatment thereof, by administering to the subject a therapeutically effective amount of one or more of the following agents: an agent that selectively decreases CLK2 protein level or kinase activity, an agent that selectively increases Akt activity, and an agent that selectively decreases the activity of protein phosphatase 2 (PP2A) comprising B56β subunit (PP2A-B56β). These methods can also include steps of assaying the level of CLK2 protein or kinase activity, Akt activity, or PP2A-B56β activity in a sample obtained from the subject; and selecting a subject who has higher CLK2 protein level or kinase activity, lower Akt activity, or higher PP2A-B56β activity, when compared to a reference level in a healthy subject, for treatment. Also provided herein are methods of monitoring a treatment of Shank3 deficiency in a subject by assaying and comparing the Akt activities in samples obtained from the subject before, during, or after the treatment. The present disclosure also provides compositions for use in treatment of Shank3 deficiency, e.g., Phelan-McDermid syndrome, autism spectrum disorder, intellectual disability, or schizophrenia.

In one aspect, provided herein are methods of treating Shank3 deficiency in a subject in need of treatment thereof by administering a therapeutically effective amount of an agent that selectively decreases Cdc2-like kinase 2 (CLK2) protein level or kinase activity to the subject. In some embodiments, the methods of treating Shank3 deficiency include the following steps: (1) assaying CLK2 protein level or kinase activity in a sample obtained from the subject; (2) determining that the subject's CLK2 protein level or kinase activity is higher than a reference CLK2 protein level or kinase activity; and (3) administering a therapeutically effective amount of an agent that selectively decreases CLK2 protein level or kinase activity to the subject. The reference CLK2 protein level or kinase activity can be the level of CLK2 protein or kinase activity in a sample obtained from a healthy subject. In some embodiments, the CLK2 protein level or kinase activity in a sample is determined by an assay selected from a kinase assay, immunohistochemistry, Western blotting, immunofluorescent assay, radioimmunoassay (RIA), enzyme-linked immunosorbent assay (ELISA), or homogeneous time resolved fluorescence (HTRF). The agent that selectively decreases CLK2 protein level or kinase activity can be selected from a RNAi agent, an antisense oligonucleotide, a ribozyme, an aptamer, an antibody or derivative thereof, or a low molecular weight compound. In some embodiments, the agent that selectively decreases CLK2 protein level or kinase activity is a low molecular weight compound, e.g., TG003. The agent that selectively decreases CLK2 protein level or kinase activity can be administered through an oral, intravenous, intracranial, or intranasal route. The methods can also include administering a second agent that treats Shank3 deficiency, e.g., risperidone, to the subject.

In another aspect, provided herein are methods of treating Shank3 deficiency in a subject in need of treatment thereof by administering a therapeutically effective amount of an agent that selectively increases protein kinase B (PKB or Akt) activity to the subject. In some embodiments, the methods of treating Shank3 deficiency include the following steps: (1) assaying Akt activity in a sample obtained from the subject; (2) determining that the subject's Akt activity is lower than a reference Akt activity; and (3) administering a therapeutically effective amount of an agent that selectively increases Akt activity to the subject. The reference Akt activity can be the level of Akt activity in a sample obtained from a healthy subject. In some embodiments, the level of Akt activity is determined by an assay selected from a kinase assay, immunohistochemistry, Western blotting, immunofluorescent assay, radioimmunoassay (RIA), enzyme-linked immunosorbent assay (ELISA), or homogeneous time resolved fluorescence (HTRF). The agent that selectively increases Akt activity can be a low molecular weight compound or an antibody or derivative thereof. In some embodiments, the agent that selectively decreases CLK2 protein level or kinase activity is a low molecular weight compound, e.g., SC79. The agent that selectively increases Akt activity can also be an agent selected from rapamycin, insulin-like growth factor-1 (IGF-1), insulin-like growth factor-2 (IGF-2), Brain-derived neurotrophic factor (BDNF), Epidermal growth factor (EGF), CC1-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, platelet derived growth factor, or granulocyte colony-stimulating factor (G-CSF). The agent that selectively increases Akt activity can be administered through an oral, intravenous, intracranial, or intranasal route. The methods can also include administering a second agent that treats Shank3 deficiency, e.g., risperidone, to the subject.

In a further aspect, provided herein are methods of treating Shank3 deficiency in a subject in need of treatment thereof by administering a therapeutically effective amount of an agent that selectively decreases the activity of protein phosphatase 2 (PP2A) comprising B56β subunit (PP2A-B56β) to the subject. In some embodiments, the methods of treating Shank3 deficiency include the following steps: (1) assaying PP2A-B56β activity in a sample obtained from the subject; (2) determining that the subject's PP2A-B56β activity is higher than a reference PP2A-B56β activity; and (3) administering a therapeutically effective amount of an agent that selectively decreases the activity of PP2A-B56β to the subject. The reference PP2A-B56β activity can be the level of PP2A-B56β activity in a sample obtained from a healthy subject. In some embodiments, the level of PP2A-B56β activity is determined by an assay selected from a phosphatase assay, immunohistochemistry, Western blotting, immunofluorescent assay, radioimmunoassay (RIA), enzyme-linked immunosorbent assay (ELISA), or homogeneous time resolved fluorescence (HTRF). The agent that selectively decreases PP2A-B56β activity can be selected from a RNAi agent, an antisense oligonucleotide, a ribozyme, an aptamer, an antibody or derivative thereof, a low molecular weight compound, or a phosphorylation-deficient variant of B56β regulatory subunit. In some embodiments, the agent that selectively decreases CLK2 protein level or kinase activity is a low molecular weight compound, e.g., okadaic acid, calyculin A, cantharidic acid, or cantharidin. The agent that selectively decreases PP2A-B56β activity can be administered through an oral, intravenous, intracranial, or intranasal route. The methods can also include administering a second agent that treats Shank3 deficiency, e.g., risperidone, to the subject.

In another aspect, provided herein are methods of selecting a subject for treatment of Shank3 deficiency. In some embodiments, the methods include (1) assaying CLK2 protein level or kinase activity in a sample obtained from a subject; and (2) selecting a subject whose CLK2 protein level or kinase activity is higher than a reference CLK2 level or kinase activity for the treatment of Shank3 deficiency. The reference CLK2 protein level or kinase activity can be the level of CLK2 protein or kinase activity in a sample obtained from a healthy subject. In some embodiments, the CLK2 protein level or kinase activity in a sample is determined by an assay selected from a kinase assay, immunohistochemistry, Western blotting, immunofluorescent assay, radioimmunoassay (RIA), enzyme-linked immunosorbent assay (ELISA), or homogeneous time resolved fluorescence (HTRF). The methods can also include assaying the level or activity of a second protein in the sample.

In some embodiments, the methods of selecting a subject for treatment of Shank3 deficiency include (1) assaying the level of Akt activity in a sample obtained from the subject; and (2) selecting a subject whose Akt activity is lower than a reference Akt activity for the treatment of Shank3 deficiency. The reference Akt activity can be the level of Akt activity in a sample obtained from a healthy subject. In some embodiments, the level of Akt activity is determined by an assay selected from a kinase assay, immunohistochemistry, Western blotting, immunofluorescent assay, radioimmunoassay (RIA), enzyme-linked immunosorbent assay (ELISA), or homogeneous time resolved fluorescence (HTRF). The methods can also include assaying the level or activity of a second protein in the sample.

In some embodiments, the methods of selecting a subject for treatment of Shank3 deficiency include (1) assaying the level of PP2A activity in a sample obtained from the subject; and (2) selecting a subject whose PP2A activity is higher than a reference PP2A activity for the treatment of Shank3 deficiency. The reference PP2A activity can be the level of PP2A activity in a sample obtained from a healthy subject. In some embodiments, the level of PP2A activity is determined by an assay selected from a phosphatase assay, immunohistochemistry, Western blotting, immunofluorescent assay, radioimmunoassay (RIA), enzyme-linked immunosorbent assay (ELISA), or homogeneous time resolved fluorescence (HTRF). The methods can also include assaying the level or activity of a second protein in the sample.

Also provided herein are methods of monitoring a treatment of Shank3 deficiency in a subject. Such methods can include assaying and comparing the Akt activities in samples obtained from the subject before, during, or after the treatment. Elevated Akt activities in samples obtained during or after the treatment when compared to the Akt activities in samples obtained before the treatment indicates that the subject responded to the treatment being evaluated. In some embodiments, such methods include (1) assaying the level of Akt activity in a first sample obtained from the subject before the treatment to obtain a first level of Akt activity; (2) assaying the level of Akt activity in a second sample obtained from the subject during or after the treatment to obtain a second level of Akt activity; and (3) comparing the first level with the second level. The treatment of Shank3 deficiency can be selected from an agent that selectively decreases CLK2 protein level or kinase activity, an agent that selectively decreases PP2A-B56β activity, or an agent that selectively increases Akt activity.

The sample used in any of the methods described herein can be a cellular or tissue sample, e.g., a cellular or tissue sample comprising olfactory neurons obtained through nasal biopsy, induced pluripotent stem cell (iPS)-derived neurons, or cerebrospinal fluid.

Also provided herein are compositions for use in treatment of Shank3 deficiency, e.g., Phelan-McDermid syndrome, autism spectrum disorder, intellectual disability, or schizophrenia. In some embodiments, such compositions include an agent that selectively decreases CLK2 protein level or kinase activity. In some embodiments, such compositions include an agent that selectively increases Akt activity. In some embodiments, such compositions include an agent that selectively decreases PP2A-B56β activity. The compositions can also include a second agent that treats Shank3 deficiency, e.g., risperidone.

Also provided herein are agents for use in the treatment of Shank3 (SH3 and multiple ankyrin repeat domains 3) deficiency in a subject wherein the treatment comprises administering a therapeutically effective amount of an agent that: (i) selectively decreases Cdc2-like kinase 2 (CLK2) protein level or kinase activity; (ii) selectively increases protein kinase B (PKB or Akt) activity; or (iii) selectively decreases the activity of protein phosphatase 2 (PP2A) comprising B56β subunit (PP2A-B56β). Shank3 deficiency includes Phelan-McDermid syndrome, autism spectrum disorder, intellectual disability, or schizophrenia. The treatment can further comprise administering a second agent that treats Shank3 deficiency, e.g., risperidone. The agent can be administered to the subject through an oral, intravenous, intracranial, or intranasal route.

Provided herein are agents for use in the treatment of Shank3 deficiency in a subject wherein the treatment comprises the steps of: (i) assaying CLK2 protein level or kinase activity in a sample obtained from the subject, determining that the subject's CLK2 protein level or kinase activity is higher than a reference CLK2 protein level or kinase activity, and administering a therapeutically effective amount of an agent that selectively decreases CLK2 protein level or kinase activity to the subject; (ii) assaying Akt activity in a sample obtained from the subject, determining that the subject's Akt activity is lower than a reference Akt activity, and administering a therapeutically effective amount of an agent that selectively increases Akt activity to the subject; or (iii) assaying PP2A-B56β activity in a sample obtained from the subject, determining that the subject's PP2A-B56β activity is higher than a reference PP2A-B56β activity, and administering a therapeutically effective amount of an agent that selectively decreases the activity of PP2A-B56β to the subject. The reference CLK2 protein level or kinase activity, the reference Akt activity or the reference PP2A-B56β activity can be the level or activity in a sample obtained from a healthy subject. The sample can be a cellular or tissue sample comprising olfactory neurons obtained through nasal biopsy, induced pluripotent stem cell (iPS)-derived neurons or cerebral spinal fluid. The level or activity of a second protein in the sample can also be assayed. The CLK2 protein level or kinase activity, the Akt activity or the PP2A-B56β activity in a sample can be determined by an assay selected from a kinase assay or a phosphatase assay, immunohistochemistry, Western blotting, immunofluorescent assay, radioimmunoassay (RIA), enzyme-linked immunosorbent assay (ELISA), or homogeneous time resolved fluorescence (HTRF). The agent can be administered to the subject through an oral, intravenous, intracranial, or intranasal route. The treatment can further comprise administering a second agent that treats Shank3 deficiency, e.g., risperidone.

Provided herein are agents for use in the treatment of Shank3 deficiency in a subject wherein the subject is selected for treatment by: (i) assaying CLK2 protein level or kinase activity in a sample obtained from a subject, and selecting a subject whose CLK2 protein level or kinase activity is higher than a reference CLK2 level or kinase activity for the treatment of Shank3 deficiency; (ii) assaying the level of Akt activity in a sample obtained from the subject, and selecting a subject whose Akt activity is lower than a reference Akt activity for the treatment of Shank3 deficiency; or (iii) assaying PP2A-B56β activity in a sample obtained from the subject, and selecting a subject whose PP2A-B56β activity is higher than a reference PP2A-B56β activity for the treatment of Shank3 deficiency. The reference CLK2 protein level or kinase activity, the reference Akt activity or the reference PP2A-B56β activity can be the level or activity in a sample obtained from a healthy subject. The sample can be a cellular or tissue sample comprising olfactory neurons obtained through nasal biopsy, induced pluripotent stem cell (iPS)-derived neurons or cerebral spinal fluid. The level or activity of a second protein in the sample can also be assayed. The CLK2 protein level or kinase activity, the Akt activity or the PP2A-B56β activity in a sample can be determined by an assay selected from a kinase assay or a phosphatase assay, immunohistochemistry, Western blotting, immunofluorescent assay, radioimmunoassay (RIA), enzyme-linked immunosorbent assay (ELISA), or homogeneous time resolved fluorescence (HTRF).

The agents for use in the treatment of Shank3 deficiency can be (i) an agent that selectively decreases CLK2 protein level or kinase activity selected from a RNAi agent, an antisense oligonucleotide, a ribozyme, an aptamer, an antibody or derivative thereof, or a low molecular weight compound, e.g., TG003; (ii) an agent that selectively increases Akt activity selected from SC79, rapamycin, insulin-like growth factor-1 (IGF-1), insulin-like growth factor-2 (IGF-2), Brain-derived neurotrophic factor (BDNF), Epidermal growth factor (EGF), CC1-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, platelet derived growth factor, or granulocyte colony-stimulating factor (G-CSF); or (iii) an agent that selectively decreases PP2A-B56β activity selected from a RNAi agent, an antisense oligonucleotide, a ribozyme, an aptamer, an antibody or derivative thereof, a low molecular weight compound, e.g., okadaic acid, calyculin A, cantharidic acid, or cantharidin, or a phosphorylation-deficient variant of B56β regulatory subunit.

Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

The term “treat” and “treatment” refer to both therapeutic treatment and prophylactic or preventive measures, wherein the object is to prevent or slow down an undesired physiological change or disorder, such as the development of a SHANK3 deficiency disease. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The term “subject” refers to an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, “Shank3” (also known as PSAP2; PROSAP2 (proline-rich synapse-associated protein 2); SPANK-2; SCZD15; DEL22q13.3; and KIAA1650) refers to a protein encoded by the SHANK3 gene. In human, SHANK3 gene is mapped to chromosomal location 22q13.3, and the human SHANK3 genomic sequence can be found at NG_008607.2. The mRNA and amino acid sequences of human SHANK3 are available in GenBank at NM_033517.1 and NP_277052.1, respectively. Human Shank3 also encompasses proteins that have over its full length at least about 90%, 91%, 92%, 93%, 94%, 95%, 96%, 97%, 98%, 99% or 100% sequence identity with the amino acid sequence of GenBank accession number NP_277052.1. A human SHANK3 nucleic acid sequence has over its full length at least about 90%, 91%, 92%, 93%, 94%, 95%, 96%, 97%, 98%, 99% or 100% sequence identity with the nucleic acid sequence of GenBank accession numbers NG_008607.2 or NM_033517.1.

The term “Shank3 deficiency” refers to a condition where the expression of Shank3 in an affected subject is reduced or eliminated when compared to a healthy subject, for example, the level of Shank3 in an affected subject is less than two-thirds, one-half, one-third, or one-fourth of the level of Shank3 in a healthy control subject. Individuals with SHANK3 deficiency can suffer from a range of symptoms, from mild to very serious physical and behavioral characteristics. Possible symptoms include, but are not limited to, severely delayed or absent speech; mental retardation; autistic behaviors; hypotonia; increased tolerance to pain; thin, flaky toenails; ptosis; poor thermoregulation; chewing non-food items; teeth grinding; tongue thrusting; hair pulling; aversion to clothes; as well as other physical and behavioral symptoms, including autism spectrum disorders and atypical schizophrenia.

As used herein, “CLK2” (CDC2-like kinase 2) refers to a dual specificity protein kinase that phosphorylates serine/threonine and tyrosine-containing substrates. The mRNA sequences of human CLK2 isoforms are available in GenBank at NM_{—} 001294338.1, NM_001294339.1 and NM_003993.3. The amino acid sequences of human CLK2 isoforms are available in GenBank at NP_001281267.1, NP_001281268.1, and NP_003984.2. The human CLK2 gene is mapped to chromosomal location 1q21, and the genomic sequence of CLK2 gene can be found in GenBank at NC_000001.11. Human CLK2 also encompasses proteins that have over its full length at least about 90%, 91%, 92%, 93%, 94%, 95%, 96%, 97%, 98%, 99% or 100% sequence identity with the amino acid sequence of GenBank accession numbers NP_001281267.1, NP_001281268.1, or NP_003984.2. A human CLK2 nucleic acid sequence has over its full length at least about 90%, 91%, 92%, 93%, 94%, 95%, 96%, 97%, 98%, 99% or 100% sequence identity with the nucleic acid sequence of GenBank accession numbers NC_000001.11, NM_001294338.1, NM_001294339.1, or NM_003993.3.

As used herein, “PKB” (also known as AKT1, RAC, CWS6, PRKBA, PKB-ALPHA, or RAC-ALPHA), refers to protein kinase B, a serine/threonine-specific kinase. The mRNA sequences of human PKB isoforms are available in GenBank at NM_005163.2, NM_001014432.1, and NM_001014431.1. The amino acid sequences of human PKB isoforms are available in GenBank at NP_005154.2, NP_001014432.1, and NP_001014431.1. The human PKB gene is mapped to chromosomal location 14q32.32, and the genomic sequence of PKB gene can be found in GenBank at NC_000014.9. Human PKB also encompasses proteins that have over its full length at least about 90%, 91%, 92%, 93%, 94%, 95%, 96%, 97%, 98%, 99% or 100% sequence identity with the amino acid sequence of GenBank accession numbers NP_005154.2, NP_001014432.1, or NP_001014431.1. A human PKB nucleic acid sequence has over its full length at least about 90%, 91%, 92%, 93%, 94%, 95%, 96%, 97%, 98%, 99% or 100% sequence identity with the nucleic acid sequence of GenBank accession numbers NC_000014.9, NM_005163.2, NM_{—} 001014432.1, or NM_001014431.1. As used herein, the term “Akt” includes Akt1, Akt2 and Akt3. Akt2 (also known as v-akt murine thymoma viral oncogene homolog 2, PKBB, PRKBB, HIHGHH, PKBBETA, or RAC-BETA) is an important signaling molecule in the insulin signaling pathway and is required to induce glucose transport. The genomic sequence of human Akt2 gene can be found in GenBank at NG_012038.2. The mRNA sequences of human Akt2 isoforms are available in GenBank at NM_001626.5, NM_001243028.2, and NM_001243027.2. The amino acid sequences of human Akt2 isoforms are available in GenBank at NP_001617.1, NP_001229957.1, and NP_001229956.1. Human Akt2 also encompasses proteins that have over its full length at least about 90%, 91%, 92%, 93%, 94%, 95%, 96%, 97%, 98%, 99% or 100% sequence identity with the amino acid sequence of GenBank accession numbers NP_001617.1, NP_001229957.1, or NP_001229956.1. A human Akt2 nucleic acid sequence has over its full length at least about 90%, 91%, 92%, 93%, 94%, 95%, 96%, 97%, 98%, 99% or 100% sequence identity with the nucleic acid sequence of GenBank accession numbers NG_012038.2, NM_001626.5, NM_001243028.2, or NM_001243027.2. The role of Akt3 (also known as MPPH, PKBG, MPPH2, PRKBG, STK-2, PKB-GAMMA, RAC-gamma, or RAC-PK-gamma) is less clear, though it appears to be predominantly expressed in the brain. The genomic sequence of human Akt3 gene can be found in GenBank at NG_029764.1. The mRNA sequences of human Akt3 isoforms are available in GenBank at NM_181690.2, NM_005465.4, and NM_001206729.1. The amino acid sequences of human Akt3 isoforms are available in GenBank at NP_859029.1, NP_005456.1, and NP_001193658.1. Human Akt3 also encompasses proteins that have over its full length at least about 90%, 91%, 92%, 93%, 94%, 95%, 96%, 97%, 98%, 99% or 100% sequence identity with the amino acid sequence of GenBank accession numbers NP_859029.1, NP_005456.1, or NP_001193658.1. A human Akt3 nucleic acid sequence has over its full length at least about 90%, 91%, 92%, 93%, 94%, 95%, 96%, 97%, 98%, 99% or 100% sequence identity with the nucleic acid sequence of GenBank accession numbers NG_029764.1, NM_181690.2, NM_005465.4, or NM_001206729.1.

The term “PP2A-B56β,” as used herein, refers to the protein phosphatase 2 (PP2A) comprising the B56β regulatory subunit. PP2A holoenzyme is a heterotrimer that consists of a structural A subunit, the catalytic C subunit, and a regulatory B subunit. The regulatory subunit B56β of PP2A is encoded by gene PPP2R5B (also known as B56B or PR61B). The mRNA and amino acid sequences of human B56β are available in GenBank at NM_006244.3 and NP_006235.1, respectively. The human PPP2R5B gene is mapped to chromosomal location 11q12, and the genomic sequence of PPP2R5B gene can be found in GenBank at NC_000011.10. Human B56β also encompasses proteins that have over its full length at least about 90%, 91%, 92%, 93%, 94%, 95%, 96%, 97%, 98%, 99% or 100% sequence identity with the amino acid sequence of GenBank accession number NP_006235.1. A human B56β nucleic acid sequence has over its full length at least about 90%, 91%, 92%, 93%, 94%, 95%, 96%, 97%, 98%, 99% or 100% sequence identity with the nucleic acid sequence of GenBank accession numbers NC_000011.10 or NM_006244.3. The catalytic subunit of PP2A is encoded by gene PPP2CA (also known as PP2Ac, RP-C, PP2CA, or PP2Calpha). The mRNA and amino acid sequences of human PP2A catalytic subunit are available in GenBank at NM_002715.2 and NP_002706.1, respectively. The structural subunit of PP2A is encoded by gene PPP2R1A (also known as MRD36, PR65A, PP2AAALPHA, or PP2A-Aalpha). The mRNA and amino acid sequences of human PP2A subunit A are available in GenBank at NM_014225.5 and NP_055040.2, respectively.

“Activity” of a protein refers to regulatory or biochemical functions of a protein in its native cell or tissue. Examples of activity of a polypeptide include both direct activities and indirect activities. Exemplary activities of CLK2 include its role as a kinase in normal neuronal cells.

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. Antibodies can be tetramers of immunoglobulin molecules. The term “antibody,” as used herein, also includes antibody fragments. The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHEI domains, multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies).

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “RNAi agent” refer to an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target gene, and which mediates the targeted cleavage of another RNA transcript via an RNA-induced silencing complex (RISC) pathway.

The term “antisense oligonucleotide” refers to a single-stranded nucleic acid molecule having a nucleobase sequence that permits hybridization to a corresponding segment of a target nucleic acid.

The term “ribozyme,” as used herein, refers to a catalytic RNA molecule capable of cleaving RNA substrates. Ribozyme specificity is dependent on complementary RNA-RNA interactions.

The term “aptamer,” as used herein, refers to an oligonucleotide or polypeptide molecule that, through its ability to adopt a specific three dimensional conformation, binds to and has an antagonizing or inhibitory effect on a protein target.

The term “low molecular weight compound” is used to describe an organic or biological compound with a molecular weight of less than or equal to 2000 Da.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A is a schematic illustration of PI3K-Akt-mTOR signaling pathway (highlighted) downstream of BDNF-activated TrkB receptor. Other principal effectors of BDNF-TrkB, ERK and PLCγ, are also shown. FIG. 1B depicts exemplary Western blot results showing that knock-down of Shank3 by shRNA (short or small hairpin RNA) in rat primary cortical neurons impairs Akt activity, but neither ERK nor PLCγ activity. FIG. 1C depicts exemplary Western blot results showing that knock-down of Shank3 in rat primary cortical neurons by two additional Shank3 shRNA vectors also impairs Akt activity, but neither ERK nor PLCγ activity. FIG. 1D depicts exemplary Western blot results showing that impaired Akt activity in the induced pluripotent stem cell (iPS)-derived neurons in two Phelan-McDermid Syndrome (PMDS) patients. FIG. 1E shows that both PMDS patients harbor short intragenic deletions within the Shank3 locus.

FIG. 2A is a schematic illustration of mass spectrometry-based profiling of relative abundance of phospho-peptides between Shank3 knock-down (shShank3) and control (shCont) neurons. FIG. 2B depicts phosphoproteomic identification of enhanced phosphorylation of B56β subunit (gene symbol PPP2r5b) in Shank3 knock-down neurons. An upregulated tryptic phosphopeptide of B5β (right) was identified in both replicates with Log2FC>1.0, in comparing Shank3 knock down (shShank3) to control (shCont) samples. Arrowheads and numbers above peptide sequence indicate potential phosphorylation sites and residue numbers in the full-length protein, respectively. FIG. 2C is a schematic illustration that shows B56β is a regulatory subunit of the heterotrimeric PP2A holoenzyme that promotes substrate specificity for PP2A-mediated dephosphorylation of Akt. FIG. 2D depicts exemplary Western blot results showing that association of the PP2A catalytic subunit (PP2Ac) with Akt is enhanced in Shank3 knock-down neurons. FIG. 2E depicts exemplary Western blot results showing that inhibiting PP2A activity by okadaic acid (OA) restores Akt activity in Shank3 knock-down (shShank3) neurons. FIG. 2F depicts exemplary Western blot results showing that overexpression of a phosphorylation-deficient variant of B56β regulatory subunit restores Akt activity in Shank3 knock-down (shShank3) neurons.

FIG. 3A is a schematic illustration that shows CLK2 phosphorylates B56β to effect homeostatic PP2A-mediated dephosphorylation of Akt. TG003 is a small molecule, ATP-competitive inhibitor of CLK2. FIG. 3B depicts exemplary Western blot results showing that upregulation of CLK2 protein level in Shank3 knock-down (shShank3) neurons when compared with control (shCont) neurons. FIG. 3C depicts exemplary Western blot results showing that BDNF rapidly augments CLK2 protein expression in control (shCont), but not Shank3 knock-down (shShank3) neurons. FIG. 3D depicts exemplary Western blot results showing that inhibition of the 26S proteasome with MG132 led to a rapid increase of CLK2 in control cells (shCont), but not in Shank3 knock-down (shShank3) neurons, suggesting impaired proteasomal degradation of CLK2 in Shank3-deficient neurons. FIG. 3E depicts exemplary Western blot results showing that attenuated ubiquitination of CLK2 in Shank3 knock-down (shShank3) neurons. FIG. 3F depicts exemplary Western blot results showing that the CLK2-inhibitor, TG003, restores Akt and rpS6 phosphorylation in Shank3 knock-down (shShank3) neurons.

FIGS. 4A and 4B are dot plots showing that no changes in CLK2 mRNA abundance were observed in Shank3 knock-down (shShank3) neurons when compared to control (shCont) neurons.

FIG. 5A is a schematic illustration showing PI3K-Akt pathways and SC79, a small molecule Akt activator. FIG. 5B depicts exemplary Western blot results showing that treatment of primary neurons with SC79 restored Akt and rpS6 phosphorylation in Shank3 knock-down neurons. FIG. 5C is a schematic illustration showing Shank3 loss of function leads to abnormally high level of CLK2 protein, which represses Akt activity via PP2A-mediated dephosphorylation. Restoring Akt phosphorylation by CLK2-inhibition (e.g., by TG003) or direct activation of Akt (e.g., with SC79) could result in beneficial effects on Shank3-deficient neurons. FIG. 5D depicts exemplary Western blot results showing that pre-treatment with a small Akt inhibitor Akti blocked BDNF-induced Akt phosphorylation in primary neurons.

FIG. 6A depicts immunohistochemistry images and the corresponding bar graph, showing that activation of Akt by SC79 treatment restores spine density in Shank3 knock-down (shShank3) neurons in hippocampal organotypic slices. FIG. 6B depicts immunohistochemistry images and the corresponding bar graph, showing that inhibition of CLK2 by TG003 treatment restores spine density in Shank3 knock-down (shShank3) neurons in hippocampal organotypic slices, in an Akt-dependent manner. FIG. 6C depicts mEPSC (miniature excitatory postsynaptic currents) recordings and corresponding bar graphs, showing that activation of Akt by SC79 treatment restores impaired synaptic function in Shank3 knock-down (shShank3) neurons. FIG. 6D depicts sEPSC (spontaneous excitatory postsynaptic currents) recordings and corresponding bar graphs, showing that inhibition of CLK2 by TG003 treatment or activation of Akt by SC79 treatment restore synaptic transmission in two PMDS patient neurons.

FIGS. 7A-7H show that knock-down of CLK2 restores dendritic spine density in Shank3-deficient neurons and Akt-activity inhibition was sufficient to reduce spine density. FIG. 7A depicts exemplary Western blot results showing the time course of Shank3 knockdown in primary neurons. Neurons were infected with lentiviruses expressing either a shRNA specific for Shank3 or a control shRNA on DIV 2, and harvested for Western blotting on the indicated day. FIG. 7B is a set of representative images of biolistically transfected hippocampal CA1 pyramidal neuron in organotypic slice culture. Dendritic spine quantification was on apical secondary dendrites (lower right). FIG. 7C and FIG. 7D are bar graphs showing knockdown of Shank3 with additional shRNAs reduced dendritic spine density of hippocampal CA1 pyramidal neurons in organotypic slice cultures, which was corrected by 24 h pre-treatment with CLK2-inhibitor TG003. Neurons were fixed for staining on DIV 14. FIG. 7E is a bar graph showing that the reduced spine density in Shank3 knockdown neurons were rescued by re-expression of non-targeted GFP-Shank3. shShank3-1 targets the 3′UTR of endogenous Shank3 mRNA and does not knockdown exogenously expressed GFP-Shank3. FIG. 7F depicts exemplary Western blot results showing CLK2 shRNAs increased Akt-phosphorylation in primary neurons. FIG. 7G shows knockdown of CLK2 by shRNA corrected spine density impairment caused by Shank3 deficiency. FIG. 7H is a bar graph showing that Akt-inhibition was sufficient to reduce dendritic spine density.

FIGS. 8A-8E illustrate generation and characterization of a Shank3 Exon 21 (C-terminal) deleted mouse model (Shank3^{Δ}C/^{Δ}C). FIG. 8A is a schematic representation of murine Shank3 protein with major domains indicated and homologous recombination-mediated targeting of Shank3 exon 21 with floxed-Neo vector for deletion. Deletion of exon 21 removes the majority of the Shank3 C-terminus. FIG. 8B shows PCR genotyping confirmation of Shank3 exon 21 deletion in Wt, Shank3^{+/ΔC}, and Shank3^{ΔC/ΔC} mice. FIG. 8C shows representative Western blot images confirming Shank3 C-terminal deletion using two antibodies. Left panel indicates loss of major Shank3 isoforms using a C-terminal specific antibody that recognizes a deleted epitope (encoded by exon 21). Right panel indicates accumulation of fast-migrating, C-terminally truncated Shank3 species in Shank3^{ΔC/ΔC} mice using an antibody recognizing the SH3 domain (see 8A). FIG. 8D shows representative Western blot images showing Shank3^{ΔC/ΔC} primary neurons exhibit upregulated CLK2 protein expression. FIG. 8E shows in vivo treatment of Shank3^{ΔC/ΔC} mice with TG003 (intraperitoneal injection with 30 mg/kg TG003) increases Akt phosphorylation.

FIGS. 9A-9K illustrate behavioral characterization of the Shank3^{ΔC/ΔC} mouse model. FIG. 9A is a set of bar graphs showing Shank3^{ΔC/ΔC} mice exhibit no change in center time, but a significant decrease in total distance traveled in 120 minutes. Shank3^{+/ΔC} mice exhibit no change in center time or total distance traveled. Data are means±SEM with one-way ANOVA, p<0.0001, Tukey's multiple comparisons test. FIG. 9B is a set of bar graphs showing Shank3^{ΔC/ΔC} and Shank3^{+/ΔC} mice show no change in time spent on open arms of the elevated zero maze. Shank3^{ΔC/ΔC} show increased total distance traveled than both wild-type and Shank3^{+/ΔC}. Data are means±SEM with one-way ANOVA, p<0.001, Tukey's multiple comparisons test. FIG. 9C is a set of bar graphs showing Shank3^{ΔC/ΔC} and Shank3^{+/ΔC} mice exhibit normal locomotor coordination in two cohorts. Mice were tested for latency (seconds) to fall on a rotarod device over three trials on a single day. FIG. 9D is a set of bar graphs showing Shank3^{ΔC/ΔC} mice exhibit increased self-grooming. Mice were isolated and self-grooming behavior was scored over a 10 minute interval. TG003 treatment reduced self-grooming in Shank3^{ΔC/ΔC} mice but did not restore it to wild type frequency. For cohort 1, data are means±SEM with one-way ANOVA, p<0.0005, Tukey's multiple comparisons test. For cohort 2, data are means±SEM with one-way ANOVA, p<0.0001, Tukey's multiple comparisons test. FIG. 9E is a bar graph showing Shank3^{ΔC/ΔC} and Shank3^{+/ΔC} mice exhibit increased avoidance behavior, assessed by decreased marble burying, that is not corrected by TG003 treatment. Mice were scored for number of marbles buried (out of total 20) over a 30 minute interval. Data are means±SEM with one-way ANOVA, p<0.0001, Tukey's multiple comparisons test. FIG. 9F is a schematic representation of the behavior test where mice were tested for social motivation and social novelty in a three-chamber arena over three phases. In phase 2, social interaction with a novel intruder is measured relative to a previously encountered object from phase 1. Phase 3 tests for social novelty with a second intruder. FIG. 9G is a bar graph showing wild type, Shank3^{+/ΔC}, and Shank3^{ΔC/ΔC} mice showed no preference for total time spent in either of the flanking chambers (independent of time spent engaging in object investigation) containing identical objects (O1), nor for time spent in the center chamber (C), during phase 1 of the three-chamber social interaction task. FIG. 9H is a bar graph showing Shank3^{ΔC/ΔC} exhibit no preference for total time spent in the chamber containing the social intruder mouse (S1) compared to the O1 chamber in phase 2 of the social interaction task. TG003 treatment of Shank3^{ΔC/ΔC} mice restores the time spent in the Si chamber to wild type levels which is significantly greater than time in the O1 chamber. Data are means±SEM with paired t test (WT p<0.0005; Shank3^{ΔC/ΔC}+TG003 p<0.05) comparing S1 to O1 chamber occupancy times within each group. FIG. 9I is a set of bar graphs showing Shank3^{ΔC/ΔC} and Shank3 mice treated with TG003 exhibit no change from wild type in total distance travelled in either phase 1 or phase 2 of the three-chamber social interaction task. FIG. 9J is a bar graph showing beneficial effect of TG003 on social investigation in Shank3^{ΔC/ΔC} is maintained 72 hours after treatment in phase 2 of the three chamber task. Data are means±SEM with paired t test (Shank3^{ΔC/ΔC}+TG003 p<0.01) comparing S1 to O1 investigation times within each group. FIG. 9K is a bar graph showing Shank3^{+/ΔC} mice exhibit no impairment in social investigation in phase 2 of the three-chamber task. Data are means±SEM with paired t test (WT p<0.0001; Shank3^{+/ΔC} p<0.05) comparing S1 to O1 investigation times within each group.

FIGS. 10A-10B show CLK2 inhibition corrects impaired social motivation in Shank3^{ΔC/ΔC} mice. FIG. 10A is a bar graph showing Shank3^{ΔC/ΔC} mice display impaired motivation for social interaction that is corrected by treatment with CLK2-inhibitor, TG003. Interaction times with the intruder mouse (S1) or the object (O1) are plotted for phase 2. Data are means±SEM with paired t tests (WT p<0.0005; Shank3^{ΔC/ΔC}+TG003 p<0.0005) comparing S1 to O1 investigation times within each group. Comparison of social interaction times across groups was by one-way ANOVA with Tukey's multiple comparisons test (p<0.0005 for differences amongst group means). FIG. 10B is a set of bar graphs showing preference index for S1 versus O1 of interaction times calculated for each test phase. Data are means±SEM (one-way ANOVA, p<0.0001, Tukey's multiple comparisons test).

FIG. 11 is a bar graph showing IGF-1 corrects deficits in dendritic spine density in Shank3 kd neurons in an Akt-dependent manner. Hippocampal organotypic slice culture neurons were transfected with shRNA vectors and slices were treated for 24 h with 1 μg/ml IGF-1, or 1 μg/ml IGF-1 and 10 μM Akti, as indicated, prior to fixation on DIV 14.

### DETAILED DESCRIPTION

Provided herein are methods and compositions for diagnosing, treating, and monitoring treatment of Shank3 deficiency associated disorders, e.g., Phelan-McDermid syndrome, autism spectrum disorder, intellectual disability, or schizophrenia. The present invention is based, at least in part, on the discovery that Shank3 deficiency leads to impaired degradation of CLK2 (CDC2-like kinase 2) protein, which phosphorylates the protein phosphatase 2A (PP2A) regulatory subunit B56β and results in recruitment of the PP2A catalytic subunit to protein kinase B (PKB or Akt) and dephosphorylation of Akt. The present invention demonstrated restoration of Akt activation, either directly or via CLK2 or PP2A inhibition, can rescue the reduced dendritic spine density and impaired frequency of synaptic transmission in Shank3-deficient neurons. Accordingly, provided herein are methods of treating Shank3 deficiency, e.g., Phelan-McDermid syndrome, autism spectrum disorder, intellectual disability, or schizophrenia, in a subject in need of treatment thereof, by administering to the subject a therapeutically effective amount of one or more of the following agents: an agent that selectively decreases CLK2 protein level or kinase activity, an agent that selectively increases Akt activity, and an agent that selectively decreases the activity of protein phosphatase 2 (PP2A) comprising B56β subunit (PP2A-B56β). These methods can also include steps of assaying the level of CLK2 protein or kinase activity, Akt activity, or PP2A-B56β activity in a sample obtained from the subject; and selecting a subject who has higher CLK2 protein level or kinase activity, lower Akt activity, or higher PP2A-B56β activity, when compared to a reference level in a healthy subject, for treatment. Also provided herein are methods of monitoring a treatment of Shank3 deficiency in a subject by assaying and comparing the Akt activities in samples obtained from the subject before, during, or after the treatment. The present disclosure also provides compositions for use in treatment of Shank3 deficiency, e.g., Phelan-McDermid syndrome, autism spectrum disorder, intellectual disability, or schizophrenia.

Shank3 is a large scaffolding protein that plays a critical role in dendritic spine formation. Reduction of Shank3 expression results in loss of dendritic spine density in many model systems. SHANK3 haploinsufficiency, caused by chromosomal aberrations or SHANK3 locus deletions or point mutations, is regarded as causative for chromosome 22q13 deletion syndrome also known as Phelan-McDermid syndrome (PMDS). The symptoms may include delayed or absent speech, intellectual disability, and a high risk of autism spectrum disorders (ASD) (Guilmatre et al., 2014, Developmental neurobiology 74, 113-122; Jiang and Ehlers, 2013, Neuron 78: 8-27; Phelan and McDermid, 2012, Molecular syndromology 2, 186-201). De novo loss of function mutations in SHANK3 have also been associated with non-syndromic ASD and intellectual disability (Durand et al., 2007, Nat. Genet. 39 (1): 25-27; Gauthier et al., 2009, American journal of medical genetics Part B, Neuropsychiatric genetics: the official publication of the International Society of Psychiatric Genetics 150B, 421-424; Hamdan et al., 2011, American journal of human genetics 88, 306-316; Leblond et al., 2014, PLoS genetics 10, e1004580; Redin et al., 2014, Journal of medical genetics 51, 724-736), as well as Schizophrenia (Gauthier et al., 2010, Proceedings of the National Academy of Sciences of the United States of America 107, 7863-7868). Genetic ablation of Shank3 in mice yields ASD-like phenotypes including aberrant social and stereotyped behavior, learning and memory deficits, and impairments in synaptic plasticity and transmission (Bozdagi et al., 2010, Molecular Autism 1: 15; Kouser et al., 2013, The Journal of neuroscience 33: 18448-18468; Peca et al., 2011, Nature 472: 437-442; Wang et al., 2011, Human Molecular Genetics 20: 3093-3108; Yang et al., 2012, The Journal of neuroscience 32: 6525-6541). Synaptic dysfunction has also been observed in Shank3-deficient neurons from PMDS patients or following shRNA-mediated silencing (Shcheglovitov et al., 2013, Nature 503: 267-271; Verpelli et al., 2011, The Journal of Biological Chemistry 286: 34839-34850).

Pilot studies found that treatments of PMDS patients with either insulin or insulin-like growth factor-1 (IGF-1) were beneficial for associated motor, cognitive and social impairments (Kolevzon et al., 2014, Molecular Autism 5: 54; Schmidt et al., 2009, Journal of Medical Genetics 46: 217-222). IGF-1 also alleviated synaptic and motor deficits in Shank3 knock-out mice (Bozdagi et al., 2013, Molecular Autism 1: 15) and synaptic impairment in PMDS neurons (Shcheglovitov et al., 2013, Nature 503: 267-271). A major effector pathway common to both insulin and IGF-1 is PI3K-Akt-mTORC1. This highly conserved signaling module regulates cellular functions including growth, proliferation, survival, and, accordingly, impacts diverse neuronal functions. Deregulation of PI3K-Akt-mTORC1 signaling has frequently been linked to ASDs. Indeed, mutations in genes which antagonize this pathway, PTEN (Cowden Syndrome) or TSC1/TSC2 (Tuberous sclerosis), in humans yield monogenetic syndromes with high risk of autism (Goffin et al., 2001, American Journal of Medical Genetics 105, 521-524; Smalley et al., 1992, Journal of Autism and Developmental Disorders 22: 339-355). Moreover, other syndromic forms of ASD are associated with either hyperactivation (Fragile X syndrome) or attenuation (Rett and Angelman syndromes) of this pathway (Cao et al., 2013, PLoS biology 11, e1001478; Ricciardi et al., 2011, Human Molecular Genetics 20: 1182-1196; Sharma et al., 2010. The Journal of neuroscience 30: 694-702). Despite this, full mechanistic understanding at the molecular level of why these agents are therapeutic is still lacking.

Identification of Signaling Pathways Impaired by Shank3 Deficiency

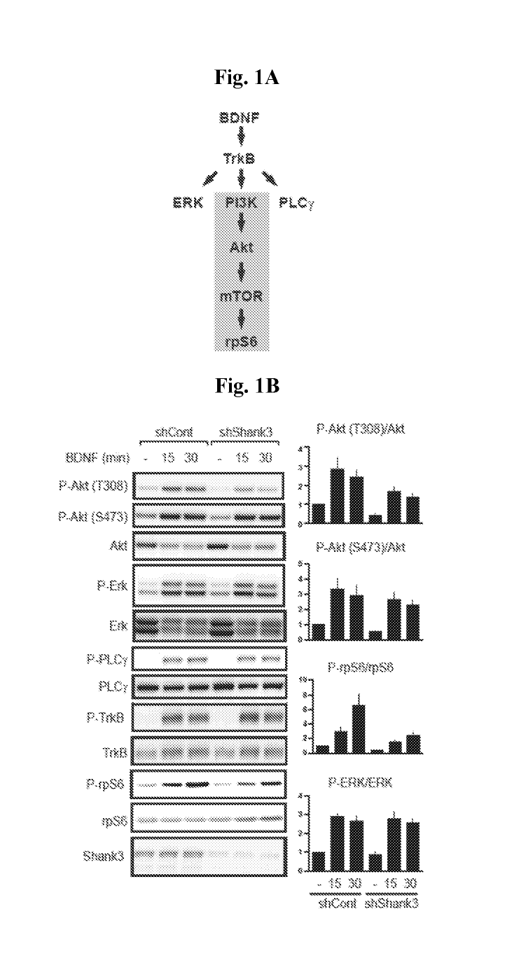

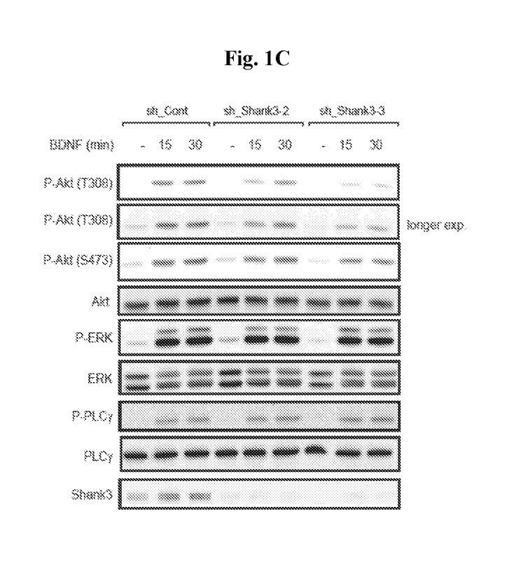

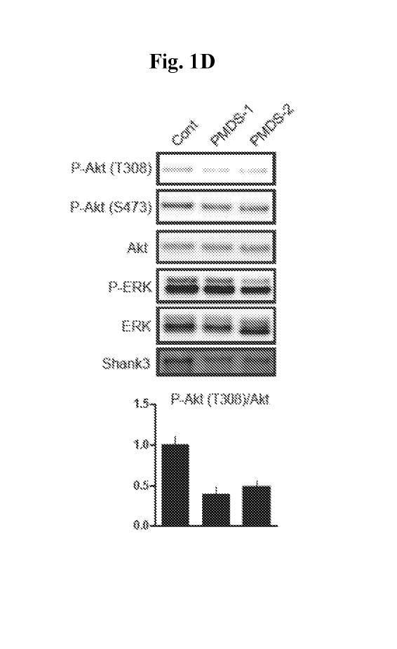

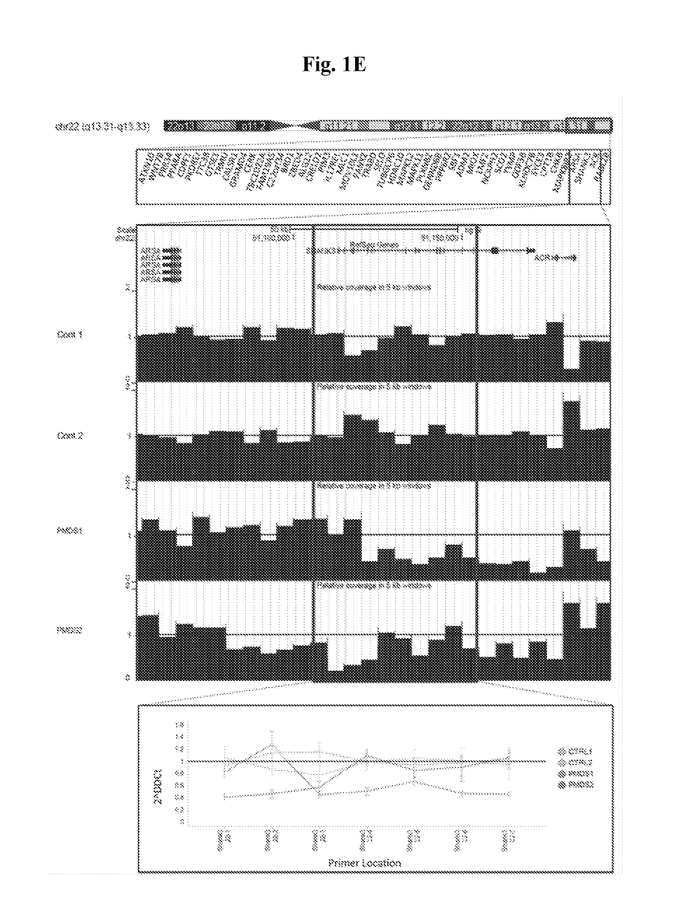

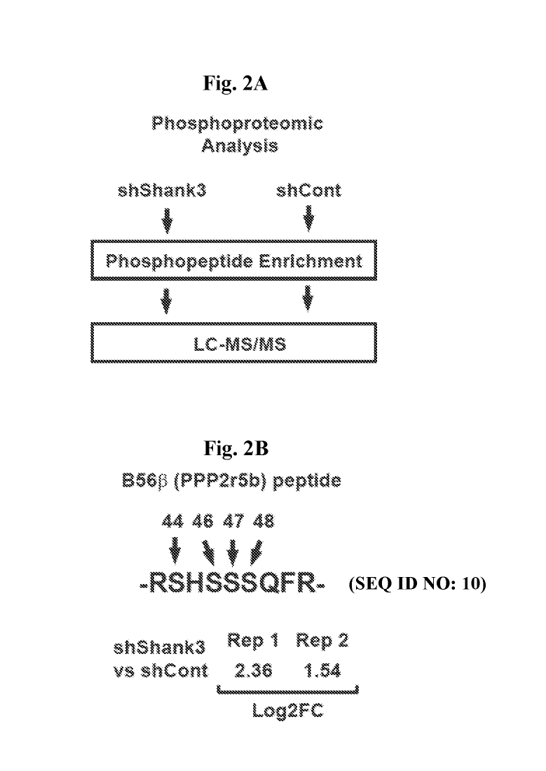

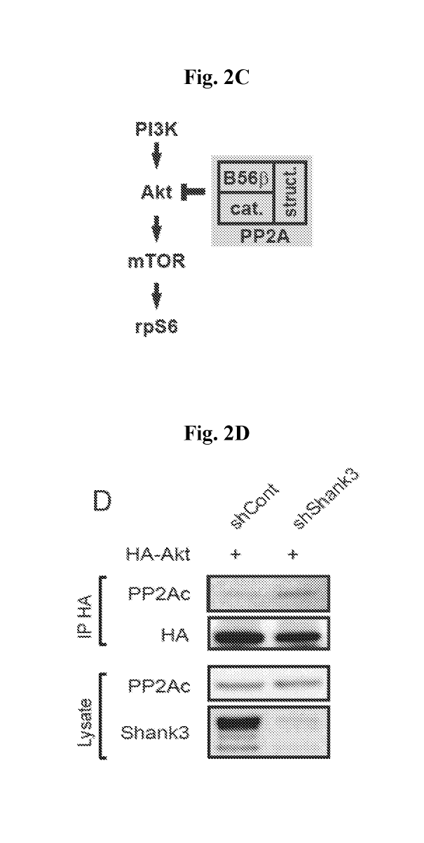

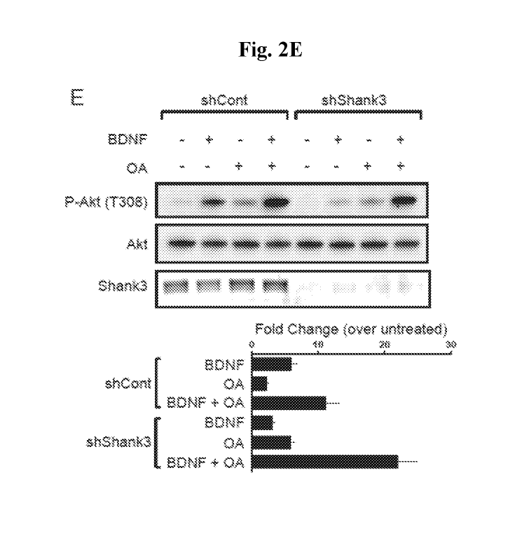

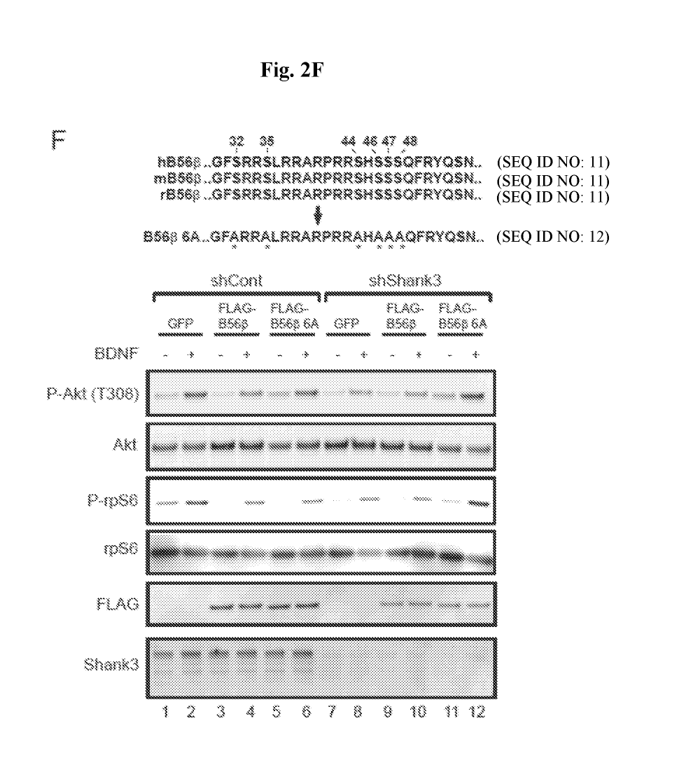

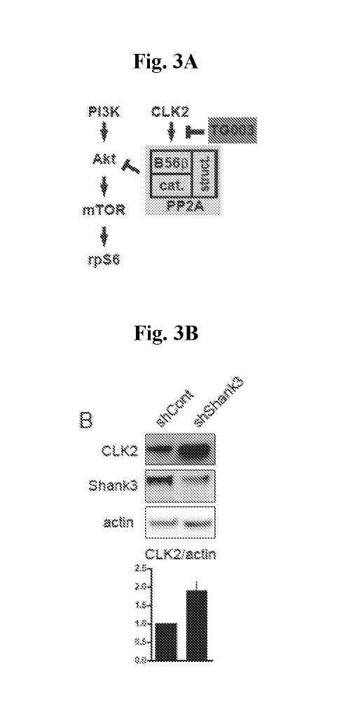

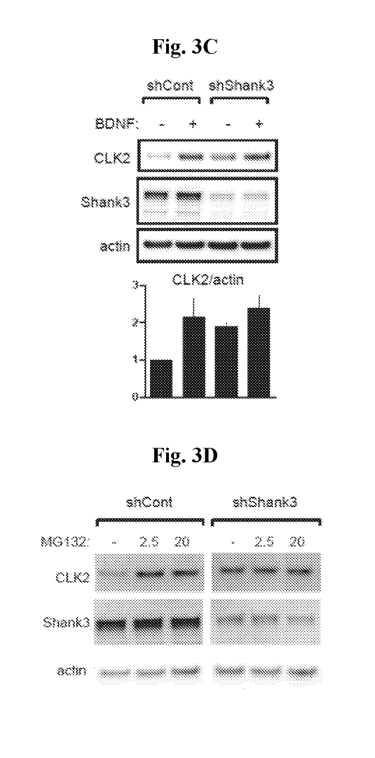

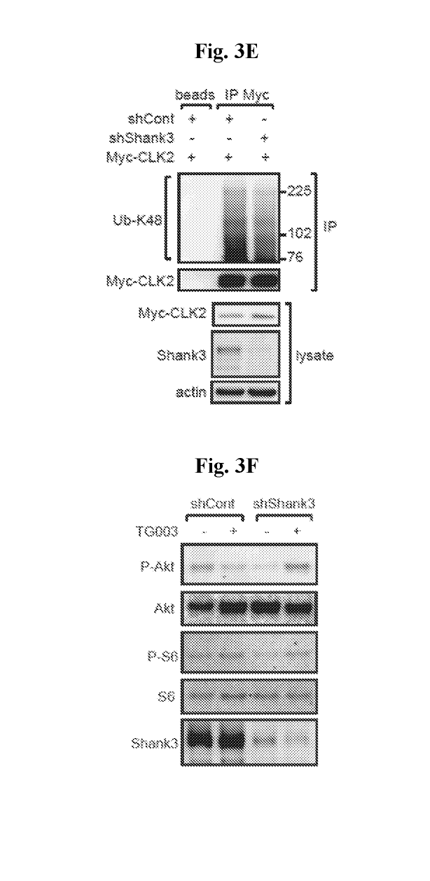

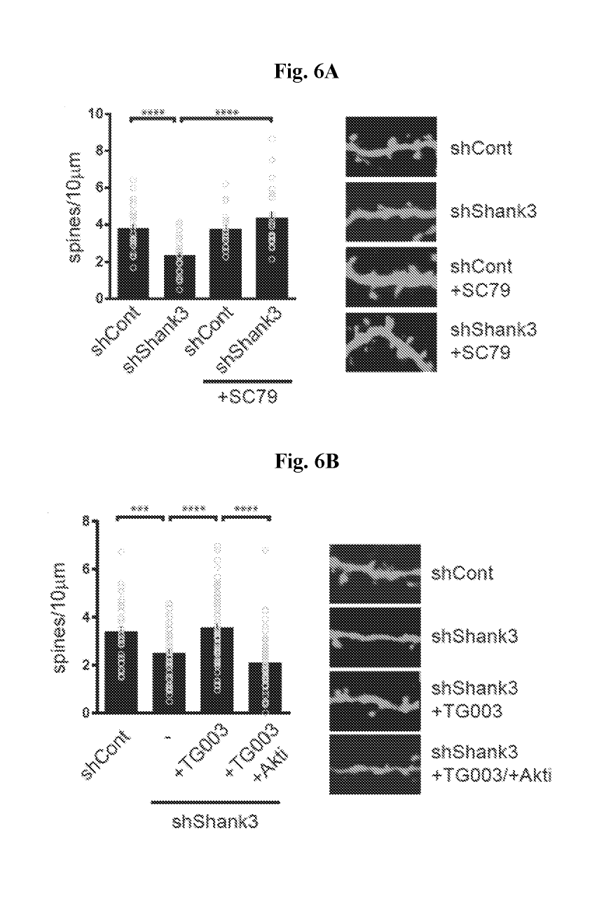

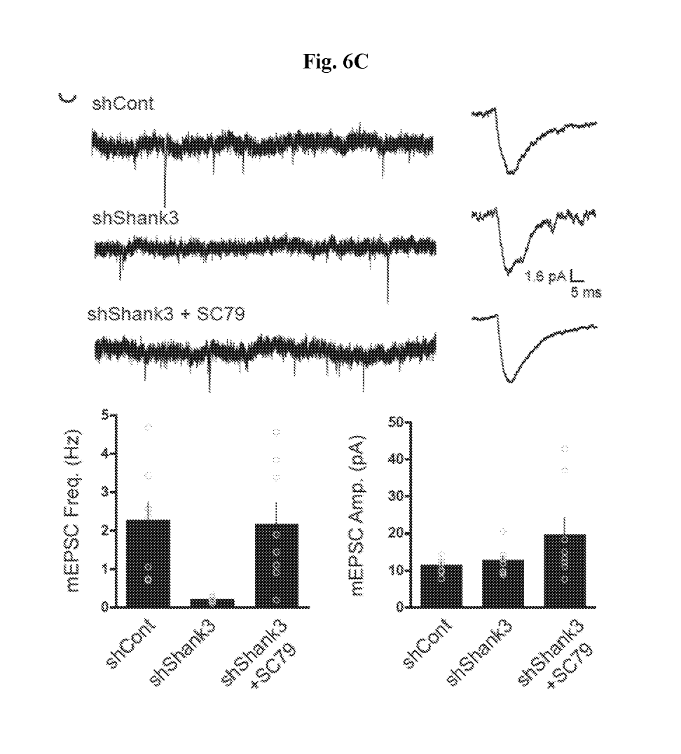

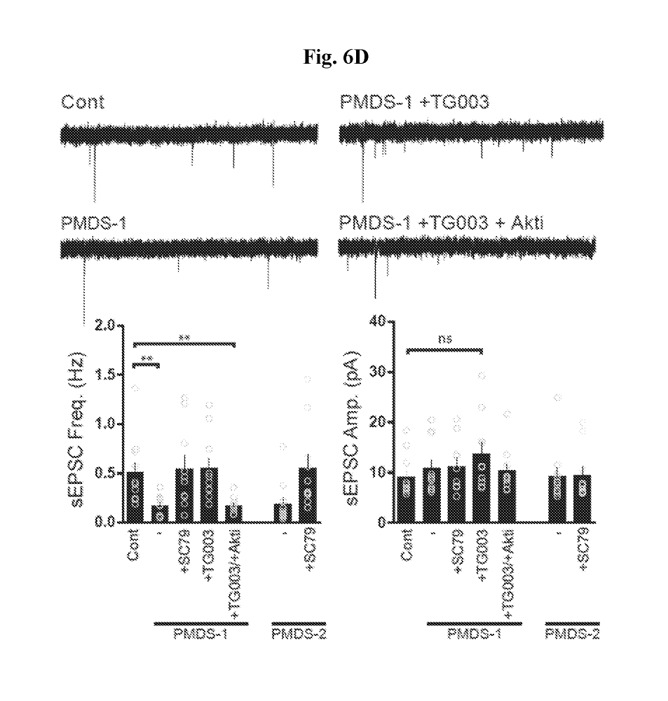

The data presented herein showed that Shank3 deficiency impairs Akt phosphorylation and activity in neurons (FIGS. 1A-1D), and identified a novel mechanism underlying this impairment: Shank3 deficiency leads to increased CLK2 protein level due to impaired ubiquitination, thereby causing aberrant steady-state expression and activation of CLK2 (FIGS. 3A-3E); and the activated CLK2 causes hyperphosphorylation of B56β, a regulatory subunit of the heterotrimeric PP2A holoenzyme, and leads to PP2A-mediated dephosphorylation and repression of Akt (FIGS. 2A-2F). Overexpression of a phosphorylation-defective B56β variant or inhibition of CLK2 with a small molecule inhibitor, e.g., TG003, restored normal Akt activity (FIG. 3F). Moreover, direct activation of Akt with a small molecule activator, e.g., SC79, or inhibition of CLK2, eliminated the neuronal impairments associated with Shank3 loss of function, e.g., diminished dendritic spine density and reduced frequency of synaptic transmission (FIGS. 6A-6D). Importantly, the beneficial effect of inhibiting CLK2 on these cellular outcomes was blocked by coincident Akt-inhibition, thereby confirming its reliance on restored Akt activity (FIGS. 6B and 6D). Thus, Shank3-deficiency and the consequent enhancement of CLK2 expression, cause a neuronal state of reduced Akt activity by favoring PP2A-dependent dephosphorylation and inactivation in opposition to upstream kinase-mediated phosphorylation.

Akt plays an important role in mammalian cellular signaling and is involved in cellular survival pathways, protein synthesis pathways, and pathways that lead to skeletal muscle hypertrophy and general tissue growth. In the developing nervous system, PKB/Akt is a critical mediator of growth factor-induced neuronal survival. PKB/Akt can be phosphorylated by phosphatidylinositol 3-kinase (PI3K), or activated in a PI3K-independent manner. Attenuated Akt activity has previously been associated with other monogenetic models of syndromic ASD, in particular MeCP2 and Ube3A deficiency in Rett and Angelman syndromes, respectively (Cao et al., 2013, PLoS biology 11: e1001478; Ricciardi et al., 2011, Human molecular genetics 20: 1182-1196). The data presented herein show that Shank3 reduction, which is causative for PMDS, also leads to impaired Akt-activation. Thus, Akt appears to be an important node whose deregulation is common to certain forms of ASD and can represent an important therapeutic target.

Akt can be dephosphoryled and repressed by protein phosphatase, e.g., protein phosphatase 2 (PP2 or PP2A). PP2A is a heterotrimer that consists of a dimeric core enzyme composed of the structural A subunit and catalytic C subunit, and a regulatory B subunit. When the PP2A core enzyme associates with the regulatory B subunit, functional PP2A holoenzyme is assembled. The structural A subunit serves as the scaffold for the formation of the heterotrimeric complex. When the structural A subunit binds to the catalytic C subunit, it alters the enzymatic activity of the catalytic C subunit, even when the regulatory B subunit is absent. While the sequences of the C and A subunits show remarkable conservation throughout eukaryotes, the sequences of the regulatory B subunits are more heterogeneous and are believed to play key roles in controlling the localization and substrate specificity of different holoenzymes. Multicellular eukaryotes express four classes of regulatory subunits: B (PR55), B′ (B56 or PR61), B″ (PR72), and B′″ (PR93/PR110), with at least 16 members in these subfamilies. In addition, accessory proteins and posttranslational modifications (such as methylation) control PP2A subunit associations and activities.

The finding that Shank3 deficiency leads to enhanced CLK2 expression provides new understanding of this signaling imbalance in PMDS. CLK2 belongs to a well conserved family of CLK kinases that phosphorylate SR (serine/arginine-rich) proteins. CLK kinases are dual-specificity kinases that phosphorylate both serine/threonine- and tyrosine-containing substrates (Nayler et al. (1997) Biochem. J. 326: 693; Ben-David et al. (1991) EMBO J. 10: 317; Howell et al. (1991) Mol. Cell. Biol. 11: 568). The amino-terminal domain of CLK2 is rich in serine and arginine, whereas the catalytic domain is very similar to CDC2, a serine/threonine protein kinase (Ben-David et al., 1991, EMBO J. 10:317-325). CLK kinases are also known as STY or LAMMER kinases (the latter based on a signature motif EHLAMMERILG (SEQ ID NO: 13) conserved between the CLK family members).

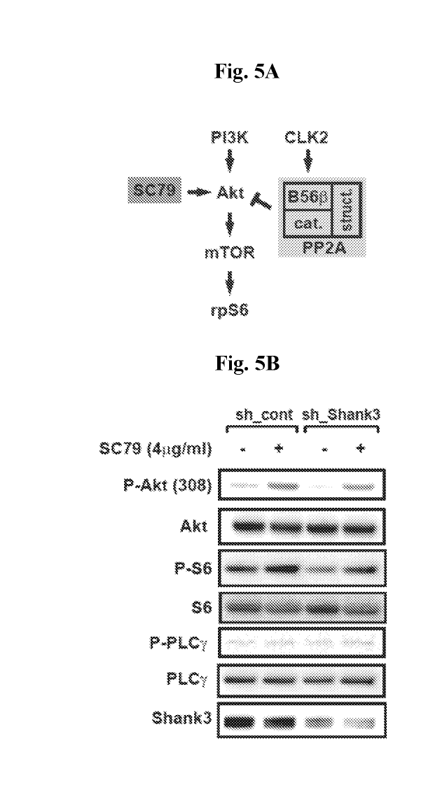

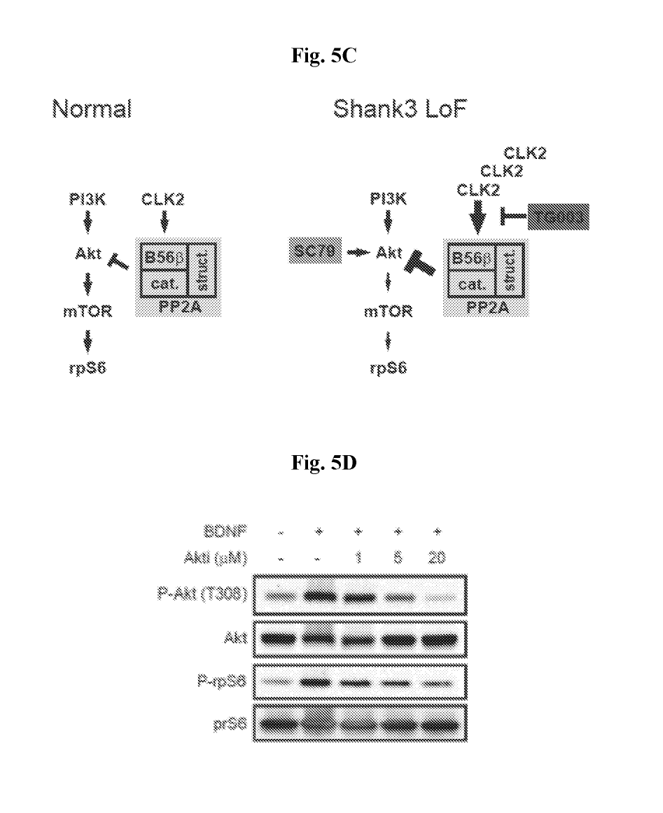

The regulation of CLK2 protein expression is complex. While cellular CLK2 protein levels are normally repressed, growth-factor stimulation leads to its Akt-mediated stabilization. This is followed by activation loop autophosphorylation which amplifies stabilization and activation, thus obviating the requirement for continued Akt-dependent signals as CLK2 kinase activity becomes self-sustaining CLK2 stabilization depends on the rapid reduction of its ubiquitination (Lee et al., 1996, The Journal of biological chemistry 271: 27299-27303; Nayler et al., 1998, The Journal of biological chemistry 273: 34341-34348; Rodgers et al., 2010, Cell metabolism 11:23-34; Rodgers et al., 2011, Molecular cell 41: 471-479). In line with this, CLK2 ubiquitination was found reduced in Shank3 knock down neurons (FIG. 3E). Furthermore, whereas proteasomal blockade sharply increased CLK2 levels in control neurons, no further increase could be elicited with the same treatment in Shank3 knock down neurons (FIG. 3D). These results suggest that the maintenance of CLK2 expression is uncoupled from ubiquitin-dependent proteasomal degradation in neurons lacking Shank3. Overnight treatment of Shank3-deficient neurons with TG003 rescued neuronal impairments in the absence of concomitant growth factor input (FIGS. 6B and 6D). This is consistent with current understanding that stabilized CLK2 is catalytically active and independent of upstream input. Therefore, constitutively high CLK2 expression in Shank3-deficient neurons would continually repress Akt, via PP2A. Restoring balanced Akt phosphorylation by CLK2-inhibition (e.g., by TG003) or direct activation of Akt (e.g., with SC79) explains the beneficial effect of these treatments on synaptic drive (FIG. 5C).

Cellular and behavioral impairments in Shank3 deficient mice, PMDS neurons, and PMDS patients, could be ameliorated with IGF-1 or insulin (Bozdagi et al., 2013, Molecular Autism 4: 9; Kolevzon et al., 2014, Molecular Autism 5: 54; Lee et al., 2011, Neuropharmacology 61, 867-879; Shcheglovitov et al., 2013, Nature 503: 267-271). Despite this progress, an understanding of the molecular mechanisms involved is conspicuously lacking. The finding that neurons from PMDS patients or following Shank3 knock down exhibit attenuated Akt activity, and are rescued from associated impairments by direct pharmacologic enhancement of Akt signaling, provides an important link to understanding signaling impairments associated with Shank3 deficiency. It may also explain why current exploratory therapeutics (e.g., IGF-1) appear to be beneficial.

The data presented herein provide the first evidence of a role for CLK2 in the nervous system. Upregulated CLK2 protein in Shank3-deficient neurons enhances B56β-dependent recruitment of PP2A catalytic subunit (PP2Ac) to Akt, leading to exaggerated Akt dephosphorylation and inactivation. IGF-1 treatment therefore conceivably restores balance in these neurons by boosting PI3K-dependent Akt phosphorylation to counteract exaggerated PP2A-mediated dephosphorylation. It is noteworthy that the Akt-activator SC79 was recently shown to restore habituation learning in an IGF-signaling impaired zebrafish model (Wolman et al., 2015, Neuron 85: 1200-1211). It is also compelling that a human chromosomal microdeletion encompassing PPP2R5B (B56β) was associated with autistic traits (Mohrmann et al., 2011, European journal of medical genetics 54, e461-464). Furthermore, a genome-wide association study identified PP2A regulation as a risk pathway common to three physchiatric disorders (Network and Pathway Analysis Subgroup of Psychiatric Genomics, 2015, Nature neuroscience 18, 199-209), while a second PP2A regulatory subunit, PPP2R5D, and a scaffold subunit, PPP2R1A, were associated with severe, undiagnosed development delay (Deciphering Developmental Disorders, 2015, Nature 519, 223-228). This suggests an important role for PP2A regulation in psychiatric and neurodevelopmental disorders.

Taken together the data presented herein provide a new mechanistic understanding of deregulated signaling downstream of Shank3 deficiency and identify new targets for therapeutic development, e.g., CLK2, PP2A-B56β, and/or Akt.

Methods of Diagnosing, Treating, and Monitoring Treatment of Shank3 Deficiency

Provided herein are methods of treating Shank3 deficiency, in a subject in need of treatment thereof, by restoring Akt activation, either directly or via CLK2 or PP2A inhibition. In some embodiments, such methods include administering a therapeutically effective amount of an agent that selectively decreases CLK2 protein level or kinase activity to the subject. In some embodiments, such methods include administering a therapeutically effective amount of an agent that selectively increases Akt activity to the subject. In some embodiments, such methods include administering a therapeutically effective amount of an agent that selectively decreases the activity of PP2A comprising B56β subunit (PP2A-B56β) to the subject. In some embodiments, such methods also include administering a second agent that treats Shank3 deficiency, e.g., risperidone, to the subject. The Shank3 deficiency can be Phelan-McDermid syndrome, autism spectrum disorder, intellectual disability, or schizophrenia.

In some embodiments, the methods of treating Shank3 deficiency in a subject in need of treatment thereof include the following steps: (1) assaying CLK2 protein level or kinase activity in a sample obtained from the subject; (2) determining that the subject's CLK2 protein level or kinase activity is higher than a reference CLK2 protein level or kinase activity; and (3) administering a therapeutically effective amount of an agent that selectively decreases CLK2 protein level or kinase activity to the subject. The reference CLK2 protein level or kinase activity can be the CLK2 protein level or kinase activity in a sample obtained from a healthy subject. The level of CLK2 protein or kinase activity in the sample can be detected and quantified by any of the means well known to those of skill in the art. These can include electrophoresis, capillary electrophoresis, high performance liquid chromatography (HPLC), thin layer chromatography (TLC), hyperdiffusion chromatography, fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry, homogeneous time resolved fluorescence (HTRF), or a kinase assay. In some embodiments, the level of CLK2 protein or kinase activity in a sample is determined by an assay selected from a kinase assay, immunohistochemistry, Western blotting, immunofluorescent assay, radioimmunoassay (RIA), enzyme-linked immunosorbent assay (ELISA), or homogeneous time resolved fluorescence (HTRF). In some embodiments, the sample is a cellular or tissue sample, e.g., a cellular or tissue sample comprising olfactory neurons obtained through nasal biopsy, induced pluripotent stem cell (iPS)-derived neurons, or cerebrospinal fluid. In some embodiments, such methods further include assaying the level or activity of a second protein in the sample.

In some embodiments, the methods of treating Shank3 deficiency in a subject in need of treatment thereof include the following steps: (1) assaying Akt activity in a sample obtained from the subject; (2) determining that the subject's Akt activity is lower than a reference Akt activity; and (3) administering a therapeutically effective amount of an agent that selectively increases Akt activity to the subject. The reference Akt activity can be the level of Akt activity in a sample obtained from a healthy subject. The level of Akt activity can be determined by a kinase assay, immunohistochemistry, Western blotting, immunofluorescent assay, radioimmunoassay (RIA), or enzyme-linked immunosorbent assay (ELISA), or homogeneous time resolved fluorescence (HTRF). In some embodiments, the sample is a cellular or tissue sample, e.g., a cellular or tissue sample comprising olfactory neurons obtained through nasal biopsy, induced pluripotent stem cell (iPS)-derived neurons, or cerebrospinal fluid. In some embodiments, such methods further include assaying the level or activity of a second protein in the sample.

In some embodiments, the methods of treating Shank3 deficiency in a subject in need of treatment thereof include one or more of the following steps: (1) assaying PP2A-B56β activity in a sample obtained from the subject; (2) determining that the subject's PP2A-B56β activity is higher than a reference PP2A-B56β activity; and (3) administering a therapeutically effective amount of an agent that selectively decreases the activity of PP2A-B56β to the subject. The reference PP2A-B56β activity can be the level of PP2A-B56β activity in a sample obtained from a healthy subject. The level of PP2A-B56β activity can be determined by a phosphatase assay, immunohistochemistry, Western blotting, immunofluorescent assay, radioimmunoassay (RIA), enzyme-linked immunosorbent assay (ELISA), or homogeneous time resolved fluorescence (HTRF). In some embodiments, the sample is a cellular or tissue sample, e.g., a cellular or tissue sample comprising olfactory neurons obtained through nasal biopsy, induced pluripotent stem cell (iPS)-derived neurons, or cerebrospinal fluid. In some embodiments, such methods further include assaying the level or activity of a second protein in the sample.

Also provided herein are methods of selecting a subject for treatment of Shank3 deficiency. In some embodiments, such methods include: (1) assaying CLK2 protein level or kinase activity in a sample obtained from a subject; and (2) selecting a subject whose CLK2 protein level or kinase activity is higher than a reference CLK2 level or kinase activity for the treatment of Shank3 deficiency. The reference CLK2 level or kinase activity can be the level of CLK2 protein or kinase activity in a sample obtained from a healthy subject. The level of CLK2 protein or kinase activity in the sample can be detected and quantified by any of the means discussed above. In some embodiments, the level of CLK2 protein or kinase activity in a sample is determined by an assay selected from an kinase assay, immunohistochemistry, Western blotting, immunofluorescent assay, radioimmunoassay (RIA), enzyme-linked immunosorbent assay (ELISA), or homogeneous time resolved fluorescence (HTRF). In some embodiments, the sample is a cellular or tissue sample, e.g., a cellular or tissue sample comprising olfactory neurons obtained through nasal biopsy, iPS-derived neurons, or cerebrospinal fluid. In some embodiments, such methods further include assaying the level or activity of a second protein in the sample.

In some embodiments, the methods of selecting a subject for treatment of Shank3 deficiency include: (1) assaying the level of Akt activity in a sample obtained from the subject; and (2) selecting a subject whose Akt activity is lower than a reference Akt activity for the treatment of Shank3 deficiency. The reference Akt activity can be the level of Akt activity in a sample obtained from a healthy subject. The level of Akt activity in a sample can be determined by a kinase assay, immunohistochemistry, Western blotting, immunofluorescent assay, radioimmunoassay (RIA), enzyme-linked immunosorbent assay (ELISA), or homogeneous time resolved fluorescence (HTRF). In some embodiments, the sample is a cellular or tissue sample, e.g., a cellular or tissue sample comprising olfactory neurons obtained through nasal biopsy, iPS-derived neurons, or cerebrospinal fluid. In some embodiments, such methods further include assaying the level or activity of a second protein in the sample.

In some embodiments, the methods of selecting a subject for treatment of Shank3 deficiency methods include: (1) assaying the level of PP2A activity in a sample obtained from the subject; (2) selecting a subject whose PP2A activity is higher than a reference PP2A activity for the treatment of Shank 3 deficiency. The reference PP2A activity can be the level of PP2A activity in a sample obtained from a healthy subject. The level of PP2A activity can be determined by a phosphatase assay, immunohistochemistry, Western blotting, immunofluorescent assay, radioimmunoassay (RIA), or enzyme-linked immunosorbent assay (ELISA), or homogeneous time resolved fluorescence (HTRF). In some embodiments, the sample is a cellular or tissue sample, e.g., a cellular or tissue sample comprising olfactory neurons obtained through nasal biopsy, iPS-derived neurons, or cerebrospinal fluid. In some embodiments, such methods further include assaying the level or activity of a second protein in the sample.

Also provided herein are methods of monitoring a treatment of Shank3 deficiency in a subject. The treatment of Shank3 deficiency can be selected from an agent that selectively decreases CLK2 protein level or kinase activity, an agent that selectively decreases PP2A-B56β activity, or an agent that selectively increases Akt activity. Such methods can include assaying and comparing the Akt activities in samples obtained from the subject before, during or after the treatment. Elevated Akt activities in samples obtained during or after the treatment when compared to the Akt activities in samples obtained before the treatment indicates that the subject responded to the treatment being evaluated. In some embodiments, such methods include (1) assaying the level of Akt activity in a first sample obtained from the subject before the treatment to obtain a first level of Akt activity; (2) assaying the level of Akt activity in a second sample obtained from the subject during or after the treatment to obtain a second level of Akt activity; and (3) comparing the first level with the second level. For example, a subject's response to an agent that selectively decreases CLK2 protein level or kinase activity can be evaluated by assaying and comparing the Akt activity in samples obtained from the subject before and after administering the agent. An increased Akt activity in a sample obtained after administering the agent when compared to the Akt activity in a sample obtained before administering the agent indicates that the subject responded to the agent that selectively decreases CLK2 protein level or kinase activity. The treatment efficacy can also be assessed based on the levels of Akt activity in samples obtained from the subject before and after administering the agent. Similarly, a subject's response to an agent that selectively decreases PP2A-B56β activity can be evaluated by assaying and comparing the Akt activity in samples obtained from the subject before and after administering the agent. An increased Akt activity in a sample obtained after administering the agent when compared to the Akt activity in a sample obtained before administering the agent indicates that the subject responded to the agent that selectively decreases PP2A-B56β activity. The treatment efficacy can also be assessed based on the levels of Akt activity in samples obtained from the subject before and after administering the agent.

Also provided herein are agents for use in the treatment of Shank3 (SH3 and multiple ankyrin repeat domains 3) deficiency in a subject wherein the treatment comprises administering a therapeutically effective amount of an agent that: (i) decreases Cdc2-like kinase 2 (CLK2) protein level or kinase activity; (ii) increases protein kinase B (PKB or Akt) activity; or (iii) decreases the activity of protein phosphatase 2 (PP2A) comprising B56β subunit (PP2A-B56β). Shank3 deficiency includes Phelan-McDermid syndrome, autism spectrum disorder, intellectual disability, or schizophrenia. The treatment can further comprise administering a second agent that treats Shank3 deficiency, e.g., risperidone. The agent can be administered to the subject through an oral, intravenous, intracranial, or intranasal route.

Provided herein are agents for use in the treatment of Shank3 deficiency in a subject wherein the treatment comprises the steps of: (i) assaying CLK2 protein level or kinase activity in a sample obtained from the subject, determining that the subject's CLK2 protein level or kinase activity is higher than a reference CLK2 protein level or kinase activity, and administering a therapeutically effective amount of an agent that selectively decreases CLK2 protein level or kinase activity to the subject; (ii) assaying Akt activity in a sample obtained from the subject, determining that the subject's Akt activity is lower than a reference Akt activity, and administering a therapeutically effective amount of an agent that selectively increases Akt activity to the subject; or (iii) assaying PP2A-B56β activity in a sample obtained from the subject, determining that the subject's PP2A-B56β activity is higher than a reference PP2A-B56β activity, and administering a therapeutically effective amount of an agent that selectively decreases the activity of PP2A-B56β to the subject. The reference CLK2 protein level or kinase activity, the reference Akt activity or the reference PP2A-B56β activity can be the level or activity in a sample obtained from a healthy subject. The sample can be a cellular or tissue sample comprising olfactory neurons obtained through nasal biopsy, induced pluripotent stem cell (iPS)-derived neurons or cerebral spinal fluid. The level or activity of a second protein in the sample can also be assayed. The CLK2 protein level or kinase activity, the Akt activity or the PP2A-B56β activity in a sample can be determined by an assay selected from a kinase assay or a phosphatase assay, immunohistochemistry, Western blotting, immunofluorescent assay, radioimmunoassay (RIA), enzyme-linked immunosorbent assay (ELISA), or homogeneous time resolved fluorescence (HTRF). The agent can be administered to the subject through an oral, intravenous, intracranial, or intranasal route. The treatment can further comprise administering a second agent that treats Shank3 deficiency, e.g., risperidone.

Provided herein are agents for use in the treatment of Shank3 deficiency in a subject wherein the subject is selected for treatment by: (i) assaying CLK2 protein level or kinase activity in a sample obtained from a subject, and selecting a subject whose CLK2 protein level or kinase activity is higher than a reference CLK2 level or kinase activity for the treatment of Shank3 deficiency; (ii) assaying the level of Akt activity in a sample obtained from the subject, and selecting a subject whose Akt activity is lower than a reference Akt activity for the treatment of Shank3 deficiency; or (iii) assaying PP2A-B56β activity in a sample obtained from the subject, and selecting a subject whose PP2A-B56β activity is higher than a reference PP2A-B56β activity for the treatment of Shank3 deficiency. The reference CLK2 protein level or kinase activity, the reference Akt activity or the reference PP2A-B56β activity can be the level or activity in a sample obtained from a healthy subject. The sample can be a cellular or tissue sample comprising olfactory neurons obtained through nasal biopsy, induced pluripotent stem cell (iPS)-derived neurons or cerebral spinal fluid. The level or activity of a second protein in the sample can also be assayed. The CLK2 protein level or kinase activity, the Akt activity or the PP2A-B56β activity in a sample can be determined by an assay selected from a kinase assay or a phosphatase assay, immunohistochemistry, Western blotting, immunofluorescent assay, radioimmunoassay (RIA), enzyme-linked immunosorbent assay (ELISA), or homogeneous time resolved fluorescence (HTRF).

An agent that selectively decreases CLK2 protein level or kinase activity can be any compound capable of selectively inhibiting the expression or kinase activity of CLK2, for example, a compound specifically inhibiting the transcription of the CLK2 gene, the maturation of CLK2 RNA, the translation of CLK2 mRNA, the posttranslational modification of the CLK2 protein, the kinase activity of the CLK2 protein, the interaction of CLK2 with a substrate, etc. An agent that selectively decreases CLK2 protein level can also refer to any agent that specifically inhibits or abrogates the normal cellular function of the CLK2 protein, either by selectively facilitating ubiquitination and degradation of the CLK2 protein, or by selective inhibition of the active kinase site, allosteric modulation of the protein structure, disruption of protein-protein interactions, or by inhibiting the transcription, translation, or stability of CLK2 protein. For example, an agent that selectively decreases CLK2 protein level or kinase activity can be a RNAi agent, an antisense oligonucleotide, a ribozyme, an aptamer, an antibody or derivative thereof, or a low molecular weight compound. In some embodiments, the CLK2 inhibitor is a low molecular weight compound, e.g., TG003.

An agent that selectively increases Akt activity refers to any compound capable of specifically activating the expression or activity of Akt (protein kinase B or PKB), for example, any compound activating the transcription of the gene, the maturation of RNA, the translation of mRNA, the posttranslational modification of the protein, the kinase activity of the protein, the interaction of Akt with a substrate, etc. An agent that selectively increases Akt activity also refers to any agent that specifically activates the normal cellular function of the Akt protein, e.g., by activation of the Akt kinase site. For example, an agent that selectively increases Akt activity can be a low molecular weight compound or an antibody or derivative thereof. In some embodiments, the Akt activator is a low molecular weight compound, e.g., SC79. Other known Akt activators include rapamycin, insulin-like growth factor-1 (IGF-1), insulin-like growth factor-2 (IGF-2), Brain-derived neurotrophic factor (BDNF), Epidermal growth factor (EGF), CC1-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, platelet derived growth factor, or granulocyte colony-stimulating factor (G-CSF).

An agent that selectively decreases PP2A-B56β activity refers to any compound capable of specifically inhibiting the expression or activity of PP2A-B56β, for example, any compound inhibiting the transcription of the gene, the maturation of RNA, the translation of mRNA, the posttranslational modification of the protein, the phosphatase activity of the protein, the interaction of PP2A-B56β with a substrate, etc. An agent that selectively decreases PP2A-B56β activity also refers to any agent that specifically inhibits or abrogates the normal cellular function of the PP2A-B56β protein, either by inhibition of the active phosphatase site, allosteric modulation of the protein structure, disruption of protein-protein interactions, or by inhibiting the transcription, translation, or stability of PP2A-B56β protein. For example, a PP2A-B56β inhibitor can be a RNAi agent, an antisense oligonucleotide, a ribozyme, an aptamer, an antibody or derivative thereof, a low molecular weight compound, or a phosphorylation-deficient variant of B56β regulatory subunit. In some embodiments, the PP2A-B56β inhibitor is a low molecular weight compound, e.g., okadaic acid, calyculin A, cantharidic acid, or cantharidin.

Antibody

The present invention provides methods of treating Shank3 deficiency in a subject in need of treatment thereof by administering to the subject a therapeutically effective amount of one or more of the following agents: an agent that selectively decreases CLK2 protein level or kinase activity, an agent that selectively increases Akt activity, and an agent that selectively decreases the activity of PP2A-B56β. One or more of those agents can be an antibody or derivative thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

The term “epitope binding domain” or “EBD” refers to portions of a binding molecule (e.g., an antibody or epitope-binding fragment or derivative thereof), that specifically interacts with (e.g., by binding, steric hindrance, stabilizing/destabilizing, spatial distribution) a binding site on a target epitope. EBD also refers to one or more fragments of an antibody that retain the ability to specifically interact with (e.g., by binding, steric hindrance, stabilizing/destabilizing, spatial distribution) a CLK2 or PP2A-B56β epitope and inhibit signal transduction. Examples of antibody fragments include, but are not limited to, an scFv, a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR).

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883).

Such single chain antibodies are also intended to be encompassed within the terms “fragment”, “epitope-binding fragment” or “antibody fragment.” These fragments are obtained using conventional techniques known to those of skill in the art, and the fragments are screened for utility in the same manner as are intact antibodies.

Antibody fragments can be incorporated into single chain molecules comprising a pair of tandem Fv segments (VH-CH1-VH-CH1) which, together with complementary light chain polypeptides, form a pair of antigen binding regions (Zapata et al., (1995) Protein Eng. 8:1057-1062; and U.S. Pat. No. 5,641,870), and also include Fab fragments, F(ab′) fragments, and anti-idiotypic (anti-Id) antibodies (including, e.g., anti-Id antibodies to antibodies of the invention), and epitope-binding fragments of any of the above.

EBDs also include single domain antibodies, maxibodies, unibodies, minibodies, triabodies, tetrabodies, v-NAR and bis-scFv, as is known in the art (see, e.g., Hollinger and Hudson, (2005) Nature Biotechnology 23: 1126-1136), bispecific single chain diabodies, or single chain diabodies designed to bind two distinct epitopes. EBDs also include antibody-like molecules or antibody mimetics, which include, but not limited to minibodies, maxybodies, Fn3 based protein scaffolds, Ankrin repeats (also known as DARpins), VASP polypeptides, Avian pancreatic polypeptide (aPP), Tetranectin, Affililin, Knottins, SH3 domains, PDZ domains, Tendamistat, Neocarzinostatin, Protein A domains, Lipocalins, Transferrin, and Kunitz domains that specifically bind epitopes, which are within the scope of the invention. Antibody fragments can be grafted into scaffolds based on polypeptides such as Fibronectin type III (Fn3) (see U.S. Pat. No. 6,703,199, which describes fibronectin polypeptide monobodies).

An isolated antibody can be a monovalent antibody, bivalent antibody, multivalent antibody, bivalent antibody, biparatopic antibody, bispecific antibody, monoclonal antibody, human antibody, recombinant human antibody, or any other type of antibody or epitope-binding fragment or derivative thereof.

The phrase “isolated antibody,” as used herein, refers to antibody that is substantially free of other antibodies having different antigenic specificities (e.g., an isolated antibody that specifically binds CLK2 is substantially free of antibodies that specifically bind antigens other than CLK2). An isolated antibody that specifically binds a target molecule may, however, have cross-reactivity to the same antigens from other species, e.g., an isolated antibody that specifically binds CLK2 may bind CLK2 molecules from other species. Moreover, an isolated antibody may be substantially free of other cellular material and/or chemicals.

The term “monovalent antibody” as used herein, refers to an antibody that binds to a single epitope on a target molecule.

The term “bivalent antibody” as used herein, refers to an antibody that binds to two epitopes on at least two identical target molecules. The bivalent antibody may also crosslink the target molecules to one another. A “bivalent antibody” also refers to an antibody that binds to two different epitopes on at least two identical target molecules.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to bivalent antibodies, trivalent antibodies, tetravalent antibodies, pentavalent antibodies, and the like, as well as bispecific antibodies and biparatopic antibodies. For example, for CLK2, the multivalent antibody (e.g., a CLK2 biparatopic antibody) has a binding moiety for two domains of CLK2, respectively.

The term “multivalent antibody” also refers to a single binding molecule that has more than one antigen-binding moiety for two separate target molecules. For example, an antibody that binds to CLK2 and a second target molecule that is not CLK2. In one embodiment, a multivalent antibody is a tetravalent antibody that has four epitope binding domains. A tetravalent molecule may be bispecific and bivalent for each binding site on that target molecule.

The term “biparatopic antibody” as used herein, refers to an antibody that binds to two different epitopes on a single target molecule. The term also includes an antibody, which binds to two domains of at least two target molecules, e.g., a tetravalent biparatopic antibody.

The term “bispecific antibody” as used herein, refers to an antibody that binds to two or more different epitopes on at least two different targets (e.g., CLK2 and a target that is not CLK2).

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody,” as used herein, includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding site” as used herein comprises an area on a target molecule to which an antibody or antigen binding fragment selectively binds.

The term “epitope” as used herein refers to any determinant capable of binding with high affinity to an immunoglobulin. An epitope is a region of an antigen that is bound by an antibody that specifically targets that antigen, and when the antigen is a protein, includes specific amino acids that directly contact the antibody. Most often, epitopes reside on proteins, but in some instances, may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics.

Generally, antibodies specific for a particular target antigen will bind to an epitope on the target antigen in a complex mixture of proteins and/or macromolecules.

As used herein, the term “affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a knock down of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can vary for other antibody isotypes. For example, high affinity binding for an IgM isotype refers to an antibody having a knock down of 10⁻⁷ M or less, or 10⁻⁸ M or less.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

RNAi Agent

The present invention provides methods of treating Shank3 deficiency in a subject in need of treatment thereof by administering to the subject a therapeutically effective amount of an agent that selectively decreases CLK2 protein level or kinase activity, or an agent that selectively decreases the activity of PP2A-B56β, wherein one or both of those agents are RNAi agents. A “RNAi agent” can be an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target gene, and which mediates the targeted cleavage of another RNA transcript via an RNA-induced silencing complex (RISC) pathway. In some embodiments, the RNAi agent is an oligonucleotide composition that activates the RISC complex/pathway. In some embodiments, the RNAi agent comprises an antisense strand sequence (antisense oligonucleotide). In some embodiments, the RNAi comprises a single strand. This single-stranded RNAi agent oligonucleotide or polynucleotide can comprise the sense or antisense strand, as described by Sioud 2005 J. Mol. Bio. 348:1079-1090, and references therein. Thus the disclosure encompasses RNAi agents with a single strand comprising either the sense or the antisense strand of an RNAi agent described herein. The use of the RNAi agent to a target gene results in a decrease of target activity, level and/or expression, e.g., a “knock-down” or “knock-out” of the target gene or target sequence.

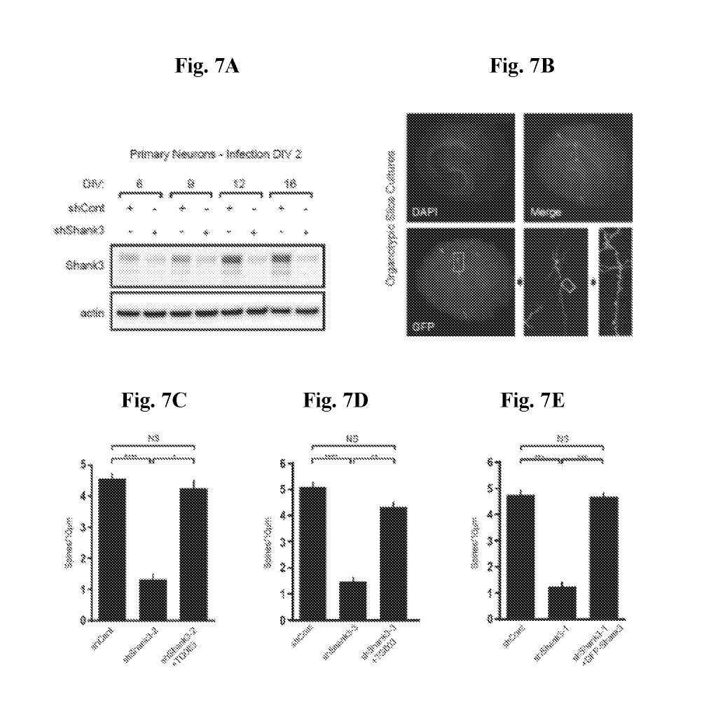

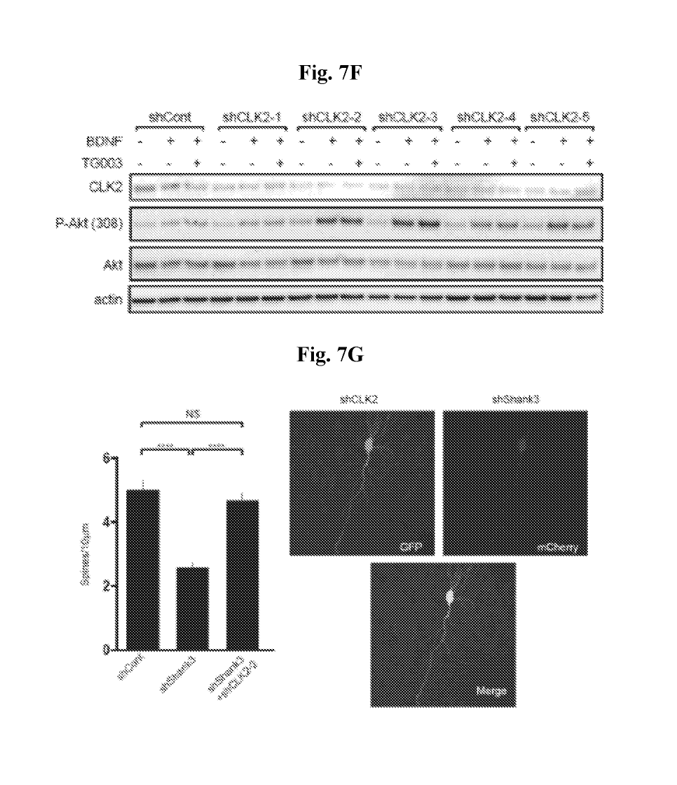

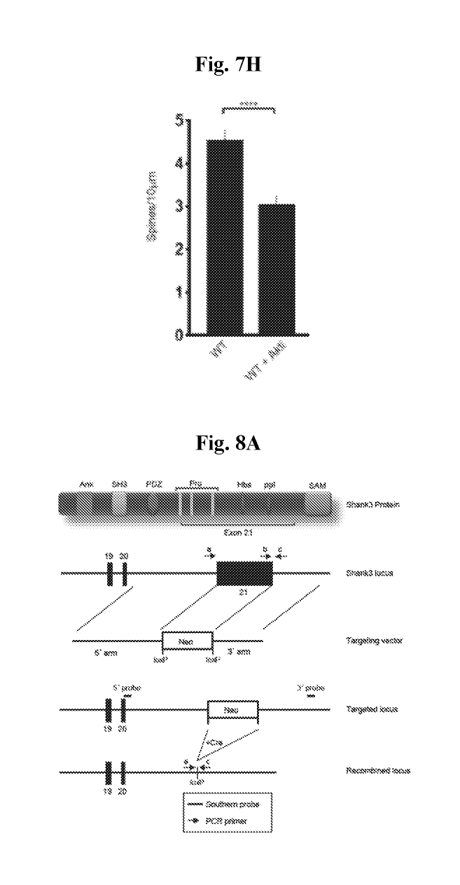

Exemplary CLK2 shRNAs are described in Example 4, e.g., shRNA having a target sequence of any of SEQ ID NOs: 5-9. As shown in FIGS. 7F and 7G, CLK2 shRNAs can increase Akt-phosphorylation in primary neurons and correct spine density impairment caused by Shank3 deficiency. Other shRNAs to CLK2 can be designed using the methods known in the art.

RNA interference is a post-transcriptional, targeted gene-silencing technique that, usually, uses double-stranded RNA (dsRNA) to degrade messenger RNA (mRNA) containing the same sequence as the dsRNA. The process of RNAi occurs naturally when ribonuclease III (Dicer) cleaves longer dsRNA into shorter fragments called siRNAs. Naturally-occurring siRNAs (small interfering RNAs) are typically about 21 to 23 nucleotides long and comprise about 19 base pair duplexes. The smaller RNA segments then mediate the degradation of the target mRNA. Dicer has also been implicated in the excision of 21- and 22-nucleotide small temporal RNAs (stRNAs) from precursor RNA of conserved structure that are implicated in translational control. Hutvagner et al. 2001, Science, 293, 834. The RNAi response also features an endonuclease complex, commonly referred to as an RNA-induced silencing complex (RISC), which mediates cleavage of single-stranded mRNA complementary to the antisense strand of the siRNA. Cleavage of the target RNA takes place in the middle of the region complementary to the antisense strand of the siRNA duplex.

“RNAi” (RNA interference) has been studied in a variety of systems. Early work in Drosophila embryonic lysates (Elbashir et al. 2001 EMBO J. 20: 6877 and Tuschl et al. International PCT Publication No. WO 01/75164) revealed certain parameters for siRNA length, structure, chemical composition, and sequence that are beneficial to mediate efficient RNAi activity. These studies have shown that 21-nucleotide siRNA duplexes are most active when containing 3′-terminal dinucleotide overhangs. Substitution of the 3′-terminal siRNA overhang nucleotides with 2′-deoxy nucleotides (2′-H) was tolerated. In addition, a 5′-phosphate on the target-complementary strand of a siRNA duplex is usually required for siRNA activity. Later work showed that a 3′-terminal dinucleotide overhang can be replaced by a 3′ end cap, provided that the 3′ end cap still allows the molecule to mediate RNA interference; the 3′ end cap also reduces sensitivity of the molecule to nucleases. See, for example, U.S. Pat. Nos. 8,097,716; 8,084,600; 8,404,831; 8,404,832; and 8,344,128. Additional later work on artificial RNAi agents showed that the strand length could be shortened, or a single-stranded nick could be introduced into the sense strand. In addition, mismatches can be introduced between the sense and anti-sense strands and a variety of modifications can be used. Any of these and various other formats for RNAi agents known in the art can be used to produce RNAi agents to CLK2 or RNAi agents to PP2A-B56β.

In some embodiments, the RNAi agent is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

Kits for RNAi synthesis are commercially available, e.g., from New England Biolabs and Ambion.

A suitable RNAi agent can be selected by any process known in the art or conceivable by one of ordinary skill in the art. For example, the selection criteria can include one or more of the following steps: initial analysis of the gene sequence and design of RNAi agents; this design can take into consideration sequence similarity across species (human, cynomolgus, mouse, etc.) and dissimilarity to other genes; screening of RNAi agents in vitro (e.g., at 10 nM in cells); determination of EC50 in HeLa cells; determination of viability of various cells treated with RNAi agents, wherein it is desired that the RNAi agent to a target molecule does not inhibit the viability of these cells; testing with human PBMC (peripheral blood mononuclear cells), e.g., to test levels of TNF-alpha to estimate immunogenicity, wherein immunostimulatory sequences are less desired; testing in human whole blood assay, wherein fresh human blood is treated with an RNAi agent and cytokine/chemokine levels are determined [e.g., TNF-alpha (tumor necrosis factor-alpha) and/or MCP1 (monocyte chemotactic protein 1)], wherein immunostimulatory sequences are less desired; determination of gene knock down in vivo using subcutaneous tumors in test animals; target gene modulation analysis, e.g., using a pharmacodynamic (PD) marker, and optimization of specific modifications of the RNAi agents.

RNAi agents can be delivered or introduced (e.g., to a cell in vitro or to a patient) by any means known in the art. “Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA may also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be achieved by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art such as electroporation and lipofection. Further approaches are described below or known in the art.

Delivery of RNAi agent to tissue can be a problem because the material must reach the target organ and must also enter the cytoplasm of target cells. RNA cannot penetrate cellular membranes, so systemic delivery of naked RNAi agent is unlikely to be successful. RNA is quickly degraded by RNAse activity in serum. For these reasons, other mechanisms to deliver RNAi agent to target cells has been devised. Methods known in the art include but are not limited to: viral delivery (retrovirus, adenovirus, lentivirus, baculovirus, AAV); liposomes (Lipofectamine, cationic DOTAP, neutral DOPC) or nanoparticles (cationic polymer, PE1), bacterial delivery (tkRNAi), and also chemical modification (LNA) of siRNA to improve stability. Xia et al. 2002 Nat. Biotechnol. 20 and Devroe et al. 2002. BMC Biotechnol. 21: 15, disclose incorporation of siRNA into a viral vector. Other systems for delivery of RNAi agents are contemplated, and the RNAi agents of the present invention can be delivered by various methods yet to be found and/or approved by the FDA or other regulatory authorities.

Liposomes have been used previously for drug delivery (e.g., delivery of a chemotherapeutic). Liposomes (e.g., cationic liposomes) are described in PCT publications W002/100435A1, W003/015757A1, and W004029213A2; U.S. Pat. Nos. 5,962,016; 5,030,453; and 6,680,068; and U.S. Patent Application 2004/0208921. A process of making liposomes is also described in W004/002453A1. Furthermore, neutral lipids have been incorporated into cationic liposomes (e.g., Farhood et al. 1995). Cationic liposomes have been used to deliver RNAi agent to various cell types (Sioud and Sorensen 2003; U.S. Patent Application 2004/0204377; Duxbury et al., 2004; Donze and Picard, 2002). Use of neutral liposomes disclosed in Miller et al. 1998, and U.S. Publ. 2003/0012812.

As used herein, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety.

Chemical transfection using lipid-based, amine-based and polymer-based techniques, is disclosed in products from Ambion Inc., Austin, Tex.; and Novagen, EMD Biosciences, Inc., an Affiliate of Merck KGaA, Darmstadt, Germany); Ovcharenko D (2003) “Efficient delivery of siRNAs to human primary cells.” Ambion TechNotes 10 (5): 15-16). Additionally, Song et al. (Nat Med. published online (Fete 10, 2003) doi: 10.1038/nm828) and others [Caplen et al. 2001 Proc. Natl. Acad. Sci. (USA), 98: 9742-9747; and McCaffrey et al. Nature 414: 34-39] disclose that liver cells can be efficiently transfected by injection of the siRNA into a mammal's circulatory system.

A variety of molecules have been used for cell-specific RNAi agent delivery. For example, the nucleic acid-condensing property of protamine has been combined with specific antibodies to deliver siRNAs. Song et al. 2005 Nat Biotch. 23: 709-717. The self-assembly PEGylated polycation polyethylenimine has also been used to condense and protect siRNAs. Schiffelers et al., 2004 Nucl. Acids Res. 32: 49, 141-110.

The siRNA-containing nanoparticles were then successfully delivered to integrin overexpressing tumor neovasculature. Hu-Lieskovan et al., 2005 Cancer Res. 65: 8984-8992.

The RNAi agents of the present invention can be delivered via, for example, Lipid nanoparticles (LNP); neutral liposomes (NL); polymer nanoparticles; double-stranded RNA binding motifs (dsRBMs); or via modification of the RNAi agent (e.g., covalent attachment to the dsRNA).

Lipid nanoparticles (LNP) are self-assembling cationic lipid based systems. These can comprise, for example, a neutral lipid (the liposome base); a cationic lipid (for siRNA loading); cholesterol (for stabilizing the liposomes); and PEG-lipid (for stabilizing the formulation, charge shielding and extended circulation in the bloodstream). The cationic lipid can comprise, for example, a headgroup, a linker, a tail and a cholesterol tail. The LNP can have, for example, good tumor delivery, extended circulation in the blood, small particles (e.g., less than 100 nm), and stability in the tumor microenvironment (which has low pH and is hypoxic). Neutral liposomes (NL) are non-cationic lipid based particles. Polymer nanoparticles are self-assembling polymer-based particles. Double-stranded RNA binding motifs (dsRBMs) are self-assembling RNA binding proteins, which will need modifications.

Ribozymes

Provided herein are methods of treating Shank3 deficiency in a subject in need of treatment thereof by administering to the subject a therapeutically effective amount of an agent that selectively decreases CLK2 protein level or kinase activity, or an agent that selectively decreases the activity of PP2A-B56β, wherein one or both of those agents are ribozymes. Ribozymes are catalytic RNA molecules capable of cleaving RNA substrates. Ribozyme specificity is dependent on complementary RNA-RNA interactions (for a review, see Cech and Bass, Annu. Rev. Biochem. 1986; 55: 599-629). Two types of ribozymes, hammerhead and hairpin, have been described. Each has a structurally distinct catalytic center. Ribozyme technology is described further in Intracellular Ribozyme Applications: Principals and Protocols, Rossi and Couture ed., Horizon Scientific Press, 1999. Ribozymes can be designed to induce catalytic cleavage of the mRNA of CLK2 or PP2A-B56β, thereby inhibiting expression of CLK2 or PP2A-B56β, respectively.

Antisense Oligonucleotides

The present invention provides methods of treating Shank3 deficiency in a subject in need of treatment thereof by administering to the subject a therapeutically effective amount of one or more of the following agents: an agent that selectively decreases CLK2 protein level or kinase activity, an agent that selectively increases Akt activity, and an agent that selectively decreases the activity of PP2A-B56β. One or more of those agents can be an antisense oligonulceotide. Antisense oligonucleotids can be DNA, RNA, a DNA-RNA chimera, or a derivative thereof. Upon hybridizing with complementary bases in an RNA or DNA molecule of interest, antisense oligonucleotids can interfere with the transcription or translation of the target gene, e.g., by inhibiting or enhancing mRNA transcription, mRNA splicing, mRNA transport, or mRNA translation or by decreasing mRNA stability. As presently used, “antisense” broadly includes RNA-RNA interactions, RNA-DNA interactions, and RNaseH mediated arrest. Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607).

Aptamers

Provided herein are methods of treating Shank3 deficiency in a subject in need of treatment thereof by administering to the subject a therapeutically effective amount of an agent that selectively decreases CLK2 protein level or kinase activity, or an agent that selectively decreases the activity of PP2A-B56β, wherein one or both of those agents are aptamers. Aptamers are usually created by selection of a large random sequence pool, but natural aptamers also exist. Inhibition of the target molecule by an aptamer may occur by binding to the target, by catalytically altering the target, by reacting with the target in a way that modifies/alters the target or the functional activity of the target, by covalently attaching to the target as a suicide inhibitor, by facilitating the reaction between the target and another inhibitory molecule. Oligonucleotide aptamers may be comprised of multiple ribonucleotide units, deoxyribonucleotide units, or a mixture of those units. Oligonucleotide aptamers may further comprise one or more modified bases, sugars, phosphate backbone units. Peptide aptamers are small, highly stable proteins that provide a high affinity binding surface for a specific target protein. They usually consist of a protein scaffold with variable peptide loops attached at both ends. The variable loop is typically composed of ten to twenty amino acids, and the scaffold can be any protein that has good solubility and compacity properties. This double structural constraint greatly increases the binding affinity of the peptide aptamer to its target protein. Aptamers can be designed to target CLK2 or PP2A-B56β protein.

Low Molecular Weight Compounds

The present invention provides methods of treating Shank3 deficiency in a subject in need of treatment thereof by administering to the subject a therapeutically effective amount of an agent that selectively decreases CLK2 protein level or kinase activity, an agent that selectively increases Akt activity, and/or an agent that selectively decreases the activity of PP2A-B56β, wherein one or more of those agents are low molecular weight compounds, e.g., a compound with a molecular weight of less than or equal to 2000 Da. For example, a low molecular weight compound that selectively activates Akt activity, e.g., SC79, or a low molecular weight compound that selectively decreases CLK2 protein level or kinase activity, e.g., TG003, can be used to treat Shank3 deficiency in any of the methods described herein.

In some embodiments, methods of treating Shank3 deficiency described herein comprise administering a therapeutically effective amount of a low molecular weight CLK2 inhibitor that selectively decreases CLK2 protein level or kinase activity. Suitable CLK2 inhibitors include TG003 ((Z)-1-(3-ethyl-5-methoxy-2,3-dihydrobenzothiazol-2-ylidene)propan-2-one) and other selective CLK2 inhibitors known in the art.

CRISPR that Inhibits CLK2 or PP2A-B56β

Clustered, regularly interspaced, short palindromic repeats (CRISPR)/CRISPR-associated (Cas) systems can be used to selectively descrease CLK2 or PP2A-B56β expression in neurons of patients with Shank3 deficiency. For example, a CRISPR-Cas system can be used to selectively edit CLK2 or PP2A-B56β gene. Such a system can include a Cas9 nuclease from S. pyogenes and an engineered single guide RNA, which includes both a crRNA (CRISPR RNA) that binds to the CLK2 or PP2A-B56β genomic DNA by base-pairing and a tracrRNA (transactivating CRISPR RNA), to direct the Cas9 nuclease to CLK2 or PP2A-B56β genomic DNA immediately 5′ to a protospacer adjacent motif (PAM), e.g., a PAM matching the sequence NGG or NAG, so that Cas9 can cleave and/or introduce mutations into the CLK2 or PP2A-B56β gene. (Sander and Joung, Nat. Biotechnol. 32(4): 347-355, 2014; Jiang et al., Nat Biotechnol 31, 233-239, 2013; Jinek et al., Elife 2, e00471, 2013; Hwang et al., Nat Biotechnol 31, 227-229, 2013; Cong et al., Science 339, 819-823, 2013; Mali et al., Science 339, 823-826, 2013c; Cho et al., Nat Biotechnol 31, 230-232, 2013; Jinek et al., Science 337, 816-821, 2012). The CRISPR-Cas system can also include a promoter to express the guide RNA, e.g., the RNA polymerase III-dependent U6 promoter or the T7 promoter. The CRISPR-Cas system can be introduced into neurons by electroporation, nucleofection, lipofectamine-mediated transfection of plasmids that express Cas9 and guide RNA, or by engineered viruses e.g., lentivirus, adenovirus, or adeno-associated viruses. The present disclosure provides use of a CRISPR/Cas system to selectively descrease CLK2 or PP2A-B56β expression for the manufacture of a medicament for treating Shank3 deficiency.

The CRISPR/Cas system has been modified for use in gene editing (silencing, enhancing or changing specific genes) in vitro (Jinek et al., Science 337, 816-821, 2012), in bacteria (Wiedenheft et al., Nature 482, 331-338, 2012; Jiang et al., Nat Biotechnol 31, 233-239, 2013) and in human cells (Cong et al., Science 339, 819-823, 2013), as well as in vivo in whole organisms such as fruit flies, zebrafish and mice (Wang et al., Cell 153, 910-918, 2013; Shen et al., Cell Res, 2013; Dicarlo et al., Nucleic Acids Res, 2013; Jiang et al., Nat Biotechnol 31, 233-239, 2013; Jinek et al., Elife 2, e00471, 2013; Hwang et al., Nat Biotechnol 31, 227-229, 2013; Cong et al., Science 339, 819-823, 2013; Mali et al., Science 339, 823-826, 2013c; Cho et al., Nat Biotechnol 31, 230-232, 2013; Gratz et al., Genetics 194(4):1029-35, 2013).

The exact arrangements of the CRISPR and structure, function and number of Cas genes and their product differ from species to species. Haft et al. 2005 PLoS Comput. Biol. 1: e60; Kunin et al. 2007. Genome Biol. 8: R61; Mojica et al. 2005. J. Mol. Evol. 60: 174-182; Bolotin et al. 2005. Microbiol. 151: 2551-2561; Pourcel et al. 2005. Microbiol. 151: 653-663; and Stern et al. 2010. Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. 2008. Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi 2013. Science 341: 833-836. One skilled in the art could design the CRISPR/Cas system to target CLK2 or PP2A-B56β using components of any known CRISPR/Cas systems.

The CRISPR/Cas system can thus be used to selectively edit a target gene such as CLK2 or PP2A-B56β (adding or deleting a basepair), e.g., introducing a premature stop and decreases expression of overexpressed CLK2 or PP2A-B56β. The CRISPR/Cas system can alternatively be used like RNA interference, turning off the target gene in a reversible fashion. In a mammalian cell, for example, the RNA can guide the Cas protein to the target promoter, sterically blocking RNA polymerases.

Artificial CRISPR/Cas systems that decrease CLK2 or PP2A-B56β expression can be generated using technology known in the art, e.g., those described in U.S. Pat. Nos. 8,697,359; 8,771,945; 8,795,965; 8,865,406; 8,871,445; 8,889,356; 8,889,418; 8,895,308; 8,906,616; 8,932,814; 8,945,839; 8,993,233; 8,999,641; 9,023,649; and U.S. Patent Publication Nos. 2015/0152398, 2014/0377868, 2014/0302563, 2014/0295557, 2014/0068797, 2013/0130248, 2011/0223638.

TALEN that Inhibits CLK2 or PP2A-B56β

TALENs are transcription activator-like effector nucleases that can be used to selectively descrease CLK2 or PP2A-B56β expression in neurons of patients with Shank3 deficiency. This disclosure provides use of a CLK2 or PP2A-B56β TALEN for the manufacture of a medicament for treating Shank3 deficiency.

TALENs can be artificially produced by fusing a TAL effector DNA binding domain to a DNA cleavage domain, e.g., a wild-type or mutated FokI endonuclease. Transcription activator-like effectors (TALEs) can be engineered to bind any desired DNA sequence, including a portion of a target gene such as CLK2 or PP2A-B56β. By combining an engineered TALE with a DNA cleavage domain, a restriction enzyme can be produced which is specific to any desired DNA sequence, including a target gene such as CLK2 or PP2A-B56β. These can then be introduced into a cell, wherein they can be used for genome editing. Boch 2011 Nature Biotech. 29: 135-6; and Boch et al. 2009 Science 326: 1509-12; Moscou et al. 2009 Science 326: 3501.

The RVDs (repeat variable diresidues) of TALE correspond to the nucleotides in their target sites in a direct, linear fashion, one RVD to one nucleotide, with some degeneracy and no apparent context dependence. In some embodiments, the RVD can comprise one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

Several TALENs with modified FokI have been made with improved cleavage specificity or activity. Cermak et al. 2011 Nucl. Acids Res. 39: e82; Miller et al. 2011 Nature Biotech. 29: 143-8; Hockemeyer et al. 2011 Nature Biotech. 29: 731-734; Wood et al. 2011 Science 333: 307; Doyon et al. 2010 Nature Methods 8: 74-79; Szczepek et al. 2007 Nature Biotech. 25: 786-793; and Guo et al. 2010 J. Mol. Biol. 200: 96.

A TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in the target gene or introduce such a defect into a wild type target gene, thus decreasing expression of a target gene such as CLK2 or PP2A-B56β.

TALENs specific to sequences in CLK2 or PP2A-B56β can be constructed using any method known in the art, e.g., the fast ligation-based automatable solid-phase high-throughput (FLASH) system described in Reyon et al., Nature Biotechnology 30, 460-465 (2012); the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeyer et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and methods using modular components as described in Zhang et al., Nat Biotechnol 29, 149-153 (2011). The TALENs can be introduced into neurons by electroporation, nucleofection, lipofectamine-mediated transfection of plasmids that express the TALENs, or by engineered viruses e.g., lentivirus, adenovirus, or adeno-associated viruses.

Zinc Finger Nuclease that Inhibits CLK2 or PP2A-B56β

ZFNs are zinc finger nucleases that can be used to selectively descrease CLK2 or PP2A-B56β expression in neurons of patients with Shank3 deficiency. This disclosure provides use of a CLK2 or PP2A-B56β ZFN for the manufacture of a medicament for treating Shank3 deficiency.

ZFNs can comprise a FokI nuclease domain (or derivative thereof) fused to a DNA-binding domain that comprises one or more zinc fingers. See Carroll et al. 2011. Genetics Society of America 188: 773-782; and Kim et al. Proc. Natl. Acad. Sci. USA 93: 1156-1160. A pair of ZFNs are required to target non-palindromic DNA sites. The two individual ZFNs must bind opposite strands of the DNA with their nucleases properly spaced apart. Bitinaite et al. 1998 Proc. Natl. Acad. Sci. USA 95: 10570-5.

Like a TALEN, a ZFN can create a double-stranded break in the DNA, which can create a frame-shift mutation if improperly repaired, leading to a decrease in the expression and amount of a target gene such as CLK2 or PP2A-B56β in a cell. ZFNs can also be used with homologous recombination to mutate, or repair defects, in a target gene such as CLK2 or PP2A-B56β.

ZFNs specific to sequences in CLK2 or PP2A-B56β can be constructed using any method known in the art, e.g., by combinatorial selection-based methods described in Maeder et al., 2008, Mol. Cell, 31:294-301; Joung et al., 2010, Nat. Methods, 7:91-92; Isalan et al., 2001, Nat. Biotechnol., 19:656-660; methods described in Cathomen et al. Mol. Ther. 16: 1200-7; Guo et al. 2010. J. Mol. Biol. 400: 96; WO 2011/017293; WO 2004/099366; U.S. Pat. No. 6,511,808; U.S. Pat. No. 6,013,453; U.S. Pat. No. 6,007,988; U.S. Pat. No. 6,503,717; U.S. 2002/0160940; Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; and Wright et al., 2006, Nat. Protoc., 1:1637-52. The ZFNs can be introduced into neurons by electroporation, nucleofection, lipofectamine-mediated transfection of plasmids that express the ZNFs, or by engineered viruses e.g., lentivirus, adenovirus, or adeno-associated viruses.

Combination Therapies

The various treatments for Shank 3 deficiency described above can be combined. For example, an agent that selectively decreases CLK2 protein level or kinase activity can be combined with an agent that selectively increases Akt activity, or an agent that selectively decreases PP2A-B56β activity. The treatment of Shank3 deficiency presented herein can be combined with other treatment partners such as the current standards of care for Shank3 deficiency, as well as potential future drugs that might be approved for Shank3 deficiency.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g., a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

Sample Preparation

Cellular or tissue samples used in the methods described herein can be obtained from a subject using any of the methods known in the art, e.g., by biopsy or surgery. For example, a cellular or tissue sample comprising olfactory neurons can be obtained through nasal biopsy or surgical resection, and a sample comprising cerebrospinal fluid can be obtained by lumbar puncture. In needle aspiration biopsy, a fine needle attached to a syringe is inserted through the skin and into the tissue of interest. The needle is typically guided to the region of interest using ultrasound or computed tomography (CT) imaging. Once the needle is inserted into the tissue, a vacuum is created with the syringe such that cells or fluid may be sucked through the needle and collected in the syringe. A tissue or cellular sample can also be removed by incisional or core biopsy. For this, a cone, a cylinder, or a tiny bit of tissue is removed from the region of interest. CT imaging, ultrasound, or an endoscope is generally used to guide this type of biopsy.

The tissue or cellular sample, may be flash frozen and stored at −80° C. for later use. The tissue or cellular sample may also be fixed with a fixative, such as formaldehyde, paraformaldehyde, or acetic acid/ethanol. The fixed tissue sample may be embedded in wax (paraffin) or a plastic resin. The embedded tissue sample (or frozen tissue sample) may be cut into thin sections. RNA or protein may also be extracted from a frozen or fixed tissue or cellular sample.

Cellular or tissue samples used in the methods described herein can contain induced pluripotent stem (iPS) cells. Dermal fibroblasts can be obtained from a subject by skin biopsy and reprogrammed into pluripotency using a CytoTune-iPS reprogramming kit (Life Technologies, Carlsbad, Calif.) according to the standard protocol. Colonies with hallmark of pluripotent morphology can be picked and subcloned multiple times on plates coated with Matrigel (BD Biosciences, San Jose, Calif.). Pluripotency can be assessed and controlled by FACS analyses using appropriate pluripotency markers, e.g., Oct3/4, Sox2, Nanog, SSEA-3 and Tra1-81 in human, and differentiation markers, e.g., SSEA-1 in human. Karyotype analyses can be performed by full-genome SNP analyses. Neuronal progenitor cells (NPCs) can be obtained by differentiating iPS cells using a modified dual SMAD inhibition as previously described (Chambers et al., 2009; Pecho-Vrieseling et al., 2014).

Pharmaceutical Compositions, Dosage, and Methods of Administration

Also provided herein are compositions, e.g., pharmaceutical compositions, for use in treatment of Shank3 deficiency. Such compositions can include one or more of the following: an agent that selectively decreases CLK2 protein level or kinase activity, an agent that selectively increases Akt activity, and an agent that selectively decreases PP2A activity. Such compositions can further include another agent that treats Shank3 deficiency, e.g., risperidone. The Shank3 deficiency can be Phelan-McDermid syndrome, autism spectrum disorder, intellectual disability, or schizophrenia.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral, e.g., intravenous, oral, intracranial, or intranasal (e.g., inhalation), intradermal, subcutaneous, transmucosal administration.

Methods of formulating suitable pharmaceutical compositions are known in the art, see, e.g., Remington: The Science and Practice of Pharmacy. 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders, for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems.

The pharmaceutical compositions can be included in a container, pack, or dispenser together with instructions for administration.

In non-limiting examples, the pharmaceutical composition containing at least one pharmaceutical agent is formulated as a liquid (e.g., a thermosetting liquid), as a component of a solid (e.g., a powder or a biodegradable biocompatible polymer (e.g., a cationic biodegradable biocompatible polymer)), or as a component of a gel (e.g., a biodegradable biocompatible polymer). In some embodiments, the at least composition containing at least one pharmaceutical agent is formulated as a gel selected from the group of an alginate gel (e.g., sodium alginate), a cellulose-based gel (e.g., carboxymethyl cellulose or carboxyethyl cellulose), or a chitosan-based gel (e.g., chitosan glycerophosphate). Additional, non-limiting examples of drug-eluting polymers that can be used to formulate any of the pharmaceutical compositions described herein include, carrageenan, carboxymethylcellulose, hydroxypropylcellulose, dextran in combination with polyvinyl alcohol, dextran in combination with polyacrylic acid, polygalacturonic acid, galacturonic polysaccharide, polysalactic acid, polyglycolic acid, tamarind gum, xanthum gum, cellulose gum, guar gum (carboxymethyl guar), pectin, polyacrylic acid, polymethacrylic acid, N-isopropylpolyacrylomide, polyoxyethylene, polyoxypropylene, pluronic acid, polylactic acid, cyclodextrin, cycloamylose, resilin, polybutadiene, N-(2-Hydroxypropyl)methacrylamide (HP MA) copolymer, maleic anhydrate-alkyl vinyl ether, polydepsipeptide, polyhydroxybutyrate, polycaprolactone, polydioxanone, polyethylene glycol, polyorganophosphazene, polyortho ester, polyvinylpyrrolidone, polylactic-co-glycolic acid (PLGA), polyanhydrides, polysilamine, poly N-vinyl caprolactam, and gellan.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Kits

Also provided herein are kits including one or more of the compositions provided herein and instructions for use. Instructions for use can include instructions for diagnosis or treatment of Shank3 deficiency. Kits as provided herein can be used in accordance with any of the methods described above, e.g., diagnosing or treating Shank3 deficiency. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

One skilled in the art will recognize many methods and materials similar or equivalent to those described herein, which could be used in the practice of the present invention. Indeed, the present invention is in no way limited to the methods and materials described.

### EXAMPLES

The invention is further described in the following examples, which do not limit the scope of the invention described in the claims.

### Example 1

### Shank3 Loss of Function impairs Akt Activity in Rat Primary Cortical Neurons and PMDS Patient Neurons

Materials and Methods

Antibodies and Reagents

The following antibodies were purchased from Cell Signaling Technology: Akt, P-Akt (T308), P-Akt (S473), Erk1/2, P-Erk1/2 (T202/Y204), PLCγ, P-PLCγ, PP2Ac subunit, P-rpS6 (S240/244), rpS6, P-TrkB (Y706/707), TrkB, and polyubiquitin K48-linkage. Shank3 antibody was obtained from Santa Cruz Biotechnology. HA-tag (rabbit) and CLK2 antibodies were from Abcam. HA-tag (HA.11, mouse) was from Covance. FLAG-tag (M2) antibody was from Sigma. GFP antibody was from Ayes Labs. TG003, Akti (Akt-VIII), and Okadaic acid were from Sigma-Aldrich. SC79 was from Millipore. MG132 was from Cell Signaling Technology. BDNF was from Bioworld.

Primary Rat Cortical Neuron Culture, Lentivirus, and Plasmid Production

E18 primary rat cortical neurons were dissected, cultured and infected 6 days after plating (DIV6) with indicated lentiviruses at MOI 10, as previously described (Proenca et al., 2013). Lentiviruses were produced by calcium phosphate transfection of HEK293T cells with packaging plasmids (Life Technologies) and transfer plasmid in 10 cm dishes. Cells were transferred to fresh medium 6 hrs. post-transfection. Four days post-transfection, the cell medium was collected and pooled from several dishes, cleared of cellular debris by 0.45 μm filtration, and concentrated 100× by centrifugation at 19,000 g for 90 min in a Beckman Ultracentrifuge using a SW28/SW32 rotor. Pellets were suspended in PBS/0.5% BSA and viral titer was determined by ELISA quantification of viral p24 antigen (Zeptometrix Corp, USA). For shRNA plasmid generation, oligonucleotides were annealed and ligated into lentiviral transfer plasmid pLKO.1-GFP vector at the AgeI/EcoRI sites. The target sequences of shRNAs that target rat and mouse Shank3 (5′-3′) are: CCACGTCACTCACAAGTTTCT (SEQ ID NO: 1), GGTTTGGAGTCTGGACTAAGC (SEQ ID NO: 2), and GGAAGTCACCAGAGGACAAGA (SEQ ID NO: 3). The latter was previously reported (Verpelli et al., 2011). A luciferase-targeting sequence was used as an shRNA control: AACTTACGCTGAGTACTTCGA (SEQ ID NO: 4).

Immunoprecipitation and Western Blotting

For immunoprecipitation (IP) of HA-tagged Akt, neurons were lysed in IP buffer (20 mM Tris-HCl pH 7.4, 3 mM EDTA, 3 mM EGTA, 150 mM NaCl, 0.5% NP-40, and protease/phosphatase inhibitor cocktails (Roche)). Lysates were cleared by centrifugation at 13,000 rpm for 10 min and lysates normalized for protein abundance were immunoprecipitated with rabbit HA-tag antibody for 1 h at 4° C. Immuno-complexes were then captured by incubation for an additional 1 hr with Protein A agarose beads (Roche). Beads were washed three times with IP buffer and bound proteins were eluted with 2×SDS-PAGE sample buffer, then boiled for 5 min. For IP and analysis of ubiquitinated Myc-Clk2, neurons were lysed in Ubiquitin IP buffer (20 mM Tris-HCl pH 7.4, 3 mM EDTA, 3 mM EGTA, 150 mM NaCl, 1% Triton X-100, protease/phosphatase inhibitor cocktails (Roche), 5 mM N-ethylmaleimide, and 3 mM iodoacetamide) containing 1% SDS. Lysates were boiled for 20 min to denature proteins and then centrifuged for 10 min at 13,000 rpm. Lysates were subsequently diluted to 0.1% SDS with Ubiquitin IP buffer and immunoprecipitated with Myc antibody for 1 h. Complexes were then captured by addition of Protein G agarose (Roche) and additional 1 h incubation. Beads were washed 3× and eluted with 2×SDS-PAGE sample buffer, then boiled for 5 min. Samples prepared for SDS-PAGE were then resolved on 4-12% Novex gels (Life Technologies), transferred to PVDF membranes, blocked in 5% low-fat milk in TBS/0.1% Tween-20, and incubated overnight with primary antibodies. After washing and 1 h incubation with secondary antibodies, proteins were visualized by Chemiluminescent detection (Amersham-GE Healthcare Life Sciences). For cell lysates prepared in RIPA buffer, as indicated in figure legends, SDS-PAGE and Western blotting was performed as above.

Genomic Real-Time PCR

Real-Time PCR analysis was conducted to detect possible Shank3 breakpoint mutations in Phelan-McDermid syndrome patient lines according to the procedure described in Bonaglia et al. The analysis was performed using the QuantStudio 12K Flex System (Life Technologies) and the Power SYBR Green PCR Master Mix (Life Technologies). Thermal cycling conditions were 95° C. for 10 min, followed by 40 cycles of 95° C. for 15 s and 60° C. for 1 min. Samples were processed in triplicate using seven couples of primers (namely 22-1 to 22-7) designed to amplify the non-repeated portions of the SHANK3 gene. For each assay, the detected Ct values were normalized using the MAPK endogenous control to obtain ΔCt values. ΔCt values for the patient samples (PMDS1, PMDS2) were further normalized to that of the wild-type SHANK3 positive control sample (CTRL1) to calculate ΔΔCt values (according to Applied Biosystems guidelines, Part Number 4387787 Rev. B). These ΔΔCt results were displayed as 2̂-ΔΔCt along with their standard deviations.

Cell Reprogramming and Differentiation

Primary human dermal fibroblasts from neonatal (Invitrogen, Carlsbad, Calif.) and adults (University of Milano, Dr. Sala) were taken for reprogramming using the CytoTune-iPS reprogramming kit (Life Technologies, Carlsbad, Calif.) according to the standard protocol. Colonies with hallmark of pluripotent morphology were readily visible between days 17 and 20 after transduction. These were picked and subcloned multiple times on plates coated with Matrigel (BD Biosciences, San Jose, Calif.) in mTeSR medium until Sendai virus RNA could no longer be detected and the morphology looked stable. Pluripotency was controlled by FACS analyses with the Human Pluripotent Stem Cell Sorting and Analysis Kit (BD Biosciences) using Oct3/4, Sox2, Nanog, SSEA-3 and Tral-81 as pluripotency markers and SSEA-1 as differentiation marker following the protocols suggested by the provider. Karyotype analyses was performed by full-genome SNP analyses by Life&Brain Gmbh (Bonn). All lines showed a normal karyotoype. Neuronal precursors were differentiated from iPS cells by using a modified dual SMAD inhibition as described earlier (Chambers et al., 2009; Pecho-Vrieseling et al., 2014). Briefly, 105 undifferentiated hiPSC were seeded in an ULA 96-well in 0.1 ml neural induction medium (20% knockout-serum replacement (Invitrogen), 0.1 mM MEM non-essential amino acids (Invitrogen), 0.1 mM 2-mercaptoethanol (Invitrogen), 75% DMEM/F11/GlutaMAX (Invitrogen Gibco); Pen/Strep, 10 ng ml bhFGF (1:1,000, Invitrogen), 10 μM SB 431542 (1:1,000) (Tocris, Bristol, UK) and 1 μM LDN 193189 (1:10,000) (Stemgent) with 10 μM Rock inhibitor to prevent apoptosis (Calbiochem, Darmstadt, Germany). Two days later 0.1 ml of fresh induction media was added to the old one. The next day 30 embryoid bodies (EB) were transferred to a 35 mm matrigel dish with induction media. At day 10 EB's were cleaned to have only neuronal rosettes left. Then the plate was trypsinized and plated on a new 35 mm matrigel plate in proliferation medium (DMEM/F12 with B27 and N2 supplements (Invitrogen) with 10 ng ml-1 bhFGF and 10 ng/ml hEGF) supplemented with 10 μM Rock inhibitor (Calbiochem). As soon as the plate was highly confluent neuronal progenitor cells (NPCs) were split by trypsinization either on matrigel plates for further amplification or frozen with 10% DMSO (vol/vol) at a concentration of 106 cells per vial and stored in liquid nitrogen. Quality control of the NPC culture was performed by using CD24, CD184 and CD44 of the Human Neural Cell Sorting Kit (BD Biosciences) according to the suggested protocol of the provider.

Statistical Analysis

All data are expressed as mean±SEM. Statistical analysis was performed by a Student's t Test (Excel, Microsoft, USA) or ANOVA, as indicated. The significance level was set at p<0.05.

Results

To determine whether Shank3 deficiency impacts the PI3K-Akt-mTOR signaling pathway (FIG. 1A, gray highlight), lentiviral-mediated delivery of shRNAs was used to knock-down Shank3 in rat primary cortical neuron cultures. Cultured rat primary cortical neurons were transduced with lentiviruses encoding control shRNA or Shank3-shRNA at six days in vitro (DIV 6) and treated with BDNF (50 ng/ml) for 15 or 30 min at 14-16 days in vitro (DIV 14-16). Cell lysates were prepared in RIPA buffer prior to resolution by SDS-PAGE and Western blotting.

Western blot analysis indicated a two-fold reduction of Akt phosphorylation at the PDK1-dependent activation loop site, T308, in the basal state (FIG. 1B). This effect was less pronounced at the mTORC2-site, S473 (FIG. 1B). Furthermore, following stimulation with brain derived neurotrophic factor (BDNF), a similar impairment of T308 phosphorylation in Akt was observed (FIG. 1B). Coincident phosphorylation of both Akt sites (T308 and S473) is necessary for full Akt kinase activation (Sarbassov et al., 2005, Science 307: 1098-1101). In agreement with this, reduced phosphorylation of ribosomal protein S6, an mTOR-dependent target downstream of Akt, was also associated with Shank3 knock down (FIG. 1B). Aside from Akt, BDNF also activates ERK and PLCγ signaling pathways downstream of its receptor, TrkB (FIG. 1A). No impaired activation of ERK and PLCγ was observed in the Shank3 knock down neurons, nor is the tyrosine-phosphorylated TrkB receptor affected in the Shank3 knock down neurons (FIG. 1B). Attenuated T308 phosphorylation of Akt and unaffected ERK or PLCγ phosphorylation, were similarly observed with two additional Shank3 shRNA vectors (FIG. 1C).

T308 phosphorylation of Akt was also reduced in human iPS-derived neurons from two PMDS patients who harbor short intragenic deletions within the Shank3 locus (FIG. 1E) and exhibit reduced Shank3 protein expression (FIG. 1D). One PMDS neuron line also exhibited slightly reduced ERK phosphorylation. The cell lysates were prepared in RIPA buffer at 8 weeks in vitro, followed by SDS-PAGE and Western blotting.

Taken together, these data demonstrate that reduced Shank3 expression impairs Akt phosphorylation and activity.

### Example 2

### Reduced Akt Activity in Shank3 Deficient Neurons is Associated with PP2A Activity

Materials and Methods

TiO2 Phosphopeptide Enrichment and LC-MS/MS Analysis

Primary cortical rat neuron cells were harvested in lysis buffer (200 mM ammonium bicarbonate pH 7.5, 8 M Urea, PhosSTOP from Roche) reduced, alkylated and digested overnight with trypsin after dilution to 2M urea. The peptides were acidified to 1% TFA, desalted on SepPak C18 cartridges and eluted with 60% acetonitrile, 0.1% TFA. Phosphopeptides were enriched from peptide mixtures using a titanium dioxide (TiO2) column. The chromatographic microcolumns were packed with TiO2 as described (Thingholm et al., 2006). Lyophilized peptides were dissolved in 80% acetonitrile, 2.5% TFA and 1M Glycolic Acid. After loading the peptide mixtures to the column, the non-phosphorylated peptides were removed with 80% acetonitrile, 2.5% TFA, 1M Glycolic Acid and 80% acetonitrile, 2.5% TFA then the phosphorylated peptides retained on the column were eluted with alkaline solution pH≥10.5 (20 μl of 25% ammonia solution in 300 μl acetonitrile and 680 μl ultra-high quality water). For LC-MS/MS, the purified phosphopeptides were resuspended in 10% formic acid and analyzed with two technical replicated each, using an EASY-nano LC system (Proxeon Biosystems, Odense, Denmark) coupled online with an LTQ-Orbitrap mass spectrometer (Thermo Scientific, Waltham, Mass.). Each sample was loaded onto a 15 cm packed in house ReproSil-Pur C18 3 μM column (75 μm inner diameter). Buffer A consisted of H2O with 0.1% formic acid and Buffer B of 100% acetonitrile with 0.1% formic acid. Peptides were separated using a gradient from 2% to 30% buffer B for 175 min, from 30% to 50% buffer B for 20 min and from 50% to 80% buffer B for 5 min (a total of 220 min at 250 nL/min). Data acquisition was done using a ‘Top 15 method’, where every full MS scan was followed by 15 data-dependent scans on the 15 most intense ions from the parent scan. Full scans were performed in the Orbitrap at 120,000 resolution with target values of 1E6 ions and 500 ms injection time, while MS/MS scans were done in the ion trap with 1E4 ions and 200 ms. Database searches were performed with Mascot Server using Uniprot database (version 3.87). Mass tolerances were set at 10 ppm for the full MS scans and at 0.8 Da for MS/MS. Label free quantification was performed on technical duplicate LC-MS runs for each sample using Progenesis LC-MS (Nonlinear Dynamics Software). The peptide intensities were normalized across all LS/MS runs by Progenesis software and normalized peptide intensities were summed for each unique phosphorylated peptide with mascot score exceeding 20. These intensities were then used to calculate the log 2 fold change ratios of each unique phosphopeptide. In case of ambiguous phosphorylation site assignments, spectra were manually interpreted for confirmation localization of the phosphorylation site using Scaffold (Proteome software).

Results

To understand the mechanistic basis for the impaired Akt activation caused by reduced Shank 3 expression, an unbiased Mass Spectrometry-based phosphoproteomic analysis was deployed to identify imbalanced signaling regulating Akt activity as described above. Specifically, phosphopeptide abundance was compared between Shank3 knock down and control neurons (FIG. 2A). Strikingly, upregulated phosphorylation of B56β (gene name PPP2r5b), on a peptide harboring an important modulatory sequence, was found in Shank3 knock down neurons (FIG. 2B). B56β is a brain-enriched, regulatory (B) subunit of the phosphatase PP2A holoenzyme that defines substrate specificity and localization (McCright et al., 1996, Genomics 36: 168-170; McCright and Virshup, 1995, The Journal of biological chemistry 270: 26123-26128). When phosphorylated by CLK2, in particular on serines contained in the peptide identified here, B56β recruits the PP2A catalytic (c) and scaffold (a) subunits to Akt for holoenzyme assembly and substrate dephosphorylation (Rodgers et al., 2011, Molecular Cell 41: 471-479). This activity is dominant for Akt T308 dephosphorylation (Padmanabhan et al., 2009, Cell 136: 939-951), which is in line with our findings of impaired phosphorylation principally at that residue (FIG. 1). To validate the phosphoproteomic findings, the interaction of Akt with the PP2A catalytic subunit (PP2Ac), which is mediated by B56β phosphorylation, was examined. Primary neurons were co-transduced with shRNA and HA-Akt lentiviruses on DIV6 and harvested in IP buffer on DIV 16. Lysates were then immunoprecipitated with HA antibody, followed by Western blotting. Immunoprecipitation of HA-tagged Akt revealed an enhanced association with PP2Ac in Shank3 knock down neurons (FIG. 2D), thereby indicating that enhanced B56β activity is responsible for impaired Akt activity via augmented interaction with PP2A.

Further validation was provided by incubating neurons with the PP2A phosphatase inihibitor, okadaic acid. Cortical neurons, transduced with shRNA-expressing lentiviruses on DIV 6, were treated with 50 ng/ml BDNF or okadaic acid (100 nM) on DIV 16 and cell lysates were resolved by SDS-PAGE, followed by Western blotting. As anticipated, okadaic acid treatment, either alone or in combination with BDNF, enhanced Akt T308 phosphorylation in both control and Shank3 knock down neurons. However, the increase of Akt T308 phosphorylation in Shank3 knock down neurons was two-fold greater than the increase in non-treated control neurons, indicating an enhanced activity of PP2A in these neurons (FIG. 2E). B56β phosphorylation was directly targeted by co-expressing a previously characterized B56β variant, which lacks CLK2-dependent phosphorylation sites (B56β 6A) (Rodgers et al., 2011, Molecular Cell 41: 471-479). Flag-tagged wild type B56β, or a variant lacking phospho-serines on the indicated sites (B56β 6A), were expressed by lentiviral co-transduction with shRNA viruses on DIV 6. Neurons were treated with 50 ng/ml BDNF and harvested in RIPA buffer on DIV 16 for SDS-PAGE and Western blotting. Whereas wild type B56β had no effect, the phosphorylation defective variant restored Akt phosphorylation in Shank3 knock down neurons to control levels (FIG. 2F; compare lanes 1, 2 with 11, 12). Thus, Shank3 loss of function in primary neurons causes a cellular state of impaired Akt activity by enhanced B56β/PP2A-mediated inactivation.

### Example 3

### Aberrant CLK2 Expression and Activation in Shank3 Deficient Neurons

What causes enhanced B56β phosphorylation in Shank3 knock down neurons? B56β is directly phosphorylated by CLK2 (Rodgers et al., 2011, Molecular Cell 41: 471-479), an event that precipitates PP2A recruitment to, and desphosphorylation of, Akt (FIG. 3A). CLK2 expression level was tested in Shank3 knock down or control neurons and a two-fold increase in CLK2 protein expression was observed in Shank3 knock down neurons when compared to control neurons (FIG. 3B). In hepatocytes, CLK2 expression is rapidly upregulated by insulin-induced Akt phosphorylation. This is followed by CLK2 activation, stabilization through reduced ubiquitination, and self-sustained activity leading to homeostatic inactivation of Akt by B56β/PP2A (Rodgers et al., 2010, Cell Metabolism 11: 23-34; Rodgers et al., 2011, Molecular Cell 41: 471-479). In agreement with these reports, treatment of primary neurons with BDNF for 30 minutes induced the accumulation of CLK2 in control neurons. However, in Shank3 knock down neurons, in which CLK2 is basally elevated, BDNF-treatment elicited no significant further increase of CLK2 levels (FIG. 3C). This suggests that the regulated expression of CLK2 by ubiquitination in neurons may be lost in the absence of Shank3. Consistent with this hypothesis, inhibition of the 26S proteasome with 2.5 or 20 μM of a proteasome inhibitor MG132 for 30 minutes led to a rapid increase of CLK2 in control cells, but not in Shank3 knock down neurons (FIG. 3D), suggesting deregulated proteasomal degradation of CLK2 in Shank3-deficient neurons.

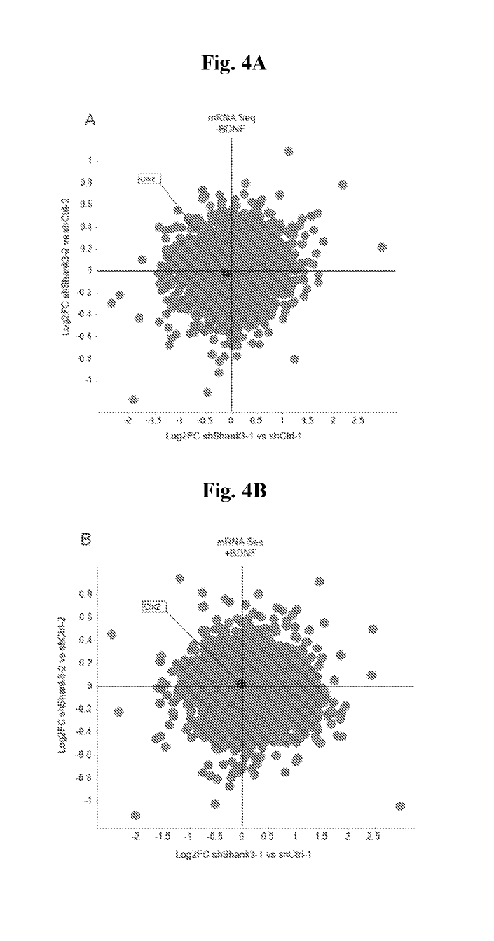

DIV6 neurons were co-transduced with shRNA and Myc-CLK2 lentiviruses. Cell lysates were prepared in IP buffer on DIV 16 and immunoprecipitated with anti-Myc antibody followed by Western blotting with a polyubiquitin antibody specific for the proteasome-targeting K48-linkage. Immunoprecipitation of overexpressed Myc-CLK2 revealed a marked decrease in ubiquitination of CLK2 in Shank3 knock down neurons (FIG. 3E). No changes in CLK2 mRNA abundance were observed (FIGS. 4A and 4B). To assay whether augmented CLK2 activity mediates attenuated Akt T308 phosphorylation in Shank3 knock down neurons, treatments with an ATP-competitive inhibitor of CLK2, TG003, were performed. DIV 16 neurons were treated with 10 μM TG003 for 60 minutes, and harvested in RIPA buffer followed by Western blotting as described above. While phosphorylation of Akt T308 in control neurons was refractory to TG003, it was restored to control levels in Shank3 knock down neurons (FIG. 3F), thereby confirming that enhanced CLK2 represses Akt activity as a consequence of reduced Shank3 expression. The lack of effect of TG003 on control neurons is not surprising given that CLK2 is maintained at low levels and lacks activity-maintaining autophosphorylation of its activation-loop in unstimulated conditions (Rodgers et al., 2010, Cell Metabolism 11: 23-34; Rodgers et al., 2011, Molecular Cell 41: 471-479).

As a whole, these results indicate that upregulated CLK2 in Shank3-deficient neurons results from its impaired ubiquitination, thereby causing aberrant steady-state expression and activation.

### Example 4

### Akt-Activation or CLK2-Inhibition Rescues Synaptic Deficits in Shank3 Deficient and PMDS Neurons

Materials and Methods

Mice and Organotypic Slice Cultures

Wilde type (C57B1/6) mice were housed in a temperature-controlled room and maintained on a 12 hr light/dark cycle. Food and water were available ad libitum and experiments were carried out in accordance with the local authorization guidelines for the care and use of laboratory animals. Slice cultures were established according to the procedure described by Stoppini and colleagues (Galimberti et al., 2006; Stoppini et al., 1991). Finally, slices were selected, placed on Millicel (Millipore, PICM03050) and cultured in 6-well dishes at 35° C. and 5% CO2 in 1 ml of culture medium. For organotypic slice cultures, brains of P6-P9 transgenic mice were dissected in cold MEM (GIBCO) medium, and hippocampal coronal sections of 400 μm were obtained with a tissue chopper (McIlwain). Slices were selected, placed on Millicell (Millipore, PICM03050) and cultured in 6-well dishes at 35° C. and 5% CO₂ in the presence of 1 ml of medium. The entire slice isolation procedure took about 30 min. The culture medium was exchanged every third day. Treatments were performed in fresh culture medium for the indicated time periods.

Biolistic Transfection

Brain slices were transfected with plasmids encoding shCont, and shShank3 using helios gene gun system (Bio-Rad Laboratories, #165-2431) as previously described (Proenca et al., 2013). Subsequently, slices were fixed, stained, mounted, and analyzed following the protocols described below in the immunohistochemistry and microscopy paragraphs.

Immunohistochemistry

Slices were fixed for 10 minutes in 4% PFA, washed in PBS and blocked for 4 hr at room temperature in 0.3% Triton X-100 20% Horse Serum/PBS (blocking solution). GFP primary antibody was incubated for 24 hr at 4° C. in the blocking solution. Afterwards, slices were washed in PBS, incubated for 2 hr in 0.3% Triton X-100/PBS with Alexa Fluor® 488 Donkey anti-chicken secondary antibody (Life technologies). Finally, slices were washed in PBS, incubated 10 minutes with DAPI (Life technologies) and mounted on glass slides using ProLong mountant (Life technologies).

Electrophysiology

Organotypice slices and cell cultures were transferred from growth medium to an interface chamber containing ACSF equilibrated with 95% O2/5% CO2 containing the following (in mM): 124 NaCl, 2.7 KCl, 2 CaCl2, 1.3 MgCl2, 26 NaHCO3, 0.4 NaH2PO4, 18 glucose, 4 ascorbate. Recordings were performed with ACSF in a recording chamber at a temperature of 35° C. at a perfusion rate of 1-2 ml/min. Neurons were visually identified with infrared video microscopy using an upright microscope equipped with a 40× objective (Olympus, Tokyo, Japan). Patch electrodes (3-5 MΩ) were pulled from borosilicate glass tubing. For voltage clamp experiments to record miniature inhibitory post-synaptic currents (mIPSCs), patch electrodes were filled with a solution containing the following (in mM): 110 CsCl, 30 K-gluconate, 1.1 EGTA, 10 HEPES, 0.1 CaCl2, 4 Mg-ATP, 0.3 Na-GTP (pH adjusted to 7.3 with CsOH, 280 mOsm) and 4 N-(2,6-Dimethylphenylcarbamoylmethyl)triethylammonium bromide (QX-314; Tocris-Cookson, Ellisville, Mo.). To exclude GABAergic inputs, picrotoxin (100 μM) was added to the ACSF. Confirmation of AMPA receptor-mediated inputs was performed by adding CNQX (6-cyano-7-nitroquinoxaline-2,3-dione, 10 μM: AMPA receptor antagonist) to the ACSF. To exclude action potential dependent IPSCs in the organotypic slices, tetrodotoxin (TTX, 1 μM) was added to the ACSF.

Whole cell patch-clamp recordings were excluded if the access resistance exceeded 13 MΩ and changed more than 20% during the recordings. Data were recorded with a MultiClamp 700B (Molecular Devices) amplifier, filtered at 0.2 kHz, and digitised at 10 kHz. Data were acquired and analysed with Clampex 10.0, Clampfit 10.0 (Molecular Devices) and the Mini Analysis Program (Synaptosoft, Decatur, Ga.). All reagents for the internal and external solutions were purchased from Fluka/Sigma (Buchs, Switzerland). Glutamatergic blockers were purchased from Tocris Bioscience (Bristol, UK). TTX was from Latoxan (Valence, France).

Microscopy and Quantification

High resolution images were acquired on an upright Zeiss LSM700 confocal microscope, using a Plan-Neofluar 100×/1.3 oil immersion objective. For the analysis of dendritic spine density, confocal 3D stacks were acquired in CA1 region for each experiment. To quantify spine density, a stretch of approximately 30 μm was selected on secondary dendrites originating at the branch point from the primary dendrite. Only secondary dendrites were considered to reduce variability. Dendritic length was measured using the ImageJ plugin, Simple Neurite tracer, and spine density manually counted using the Cell Counter plugin.

Results

The above findings implicate CLK2 overexpression as a specific anomaly that is causative for attenuated Akt activity in neurons deficient for Shank3. Consequently, inhibiting CLK2 activity or directly activating Akt would restore neuronal impairments associated with Shank3 loss of function. First, a small-molecule Akt-activator, SC79, was tested for its ability to restore Akt phosphorylation (FIG. 5A). SC79 binds directly to Akt to facilitate an ‘open’ conformation of the protein that is accessible to upstream activating kinases (eg. PDK1). This obviates the need for PtdIns(3,4,5)P₃ (PIP₃) production and recruitment of Akt to the plasma membrane (Rodgers, Cell Metab. 11, 23-34, 2010). Treatment of primary neurons with SC79 restored Akt and rpS6 phosphorylation in Shank3 knock down primary neurons, thus validating its potential to rescue neuronal deficits (FIG. 5B). Decreased principal neuron dendritic spine density has frequently been observed in Shank3 loss of function models (Durand et al., 2007, Nature Genetics 39: 25-27; Peca et al., 2011, Nature 472: 437-442; Verpelli et al., 2011, The Journal of Biological Chemistry 286: 34839-34850). Knock-down of Shank3 in hippocampal organotypic slice cultures, via biolistic transfection of shRNA vectors, faithfully recapitulated this phenotype in CA1 pyramidal neurons, whereby a two-fold reduction in apical dendrite spine density resulted (FIG. 6A). Importantly, the organotypic model system is amenable to protracted ex vivo treatments (days to weeks), such as shRNA-mediated knock-down, while preserving the neuronal and synaptic architecture of the parental brain region from which it is derived (Galimberti et al., 2010, Neuron 65: 627-642; Galimberti et al., 2006, Neuron 50: 749-763). Hippocampal organotypic slices were biolistically transfected with shRNA plasmids at DIV 1 and treated on DIV 14 for 24 hr with 4 μg/ml SC79, prior to fixation and immunostaining for GFP. Spines of CA1 pyramidal neurons were quantified on apical secondary dendrites. Treatment with SC79 rescued the spine density impairment in Shank3 knock down neurons without significantly affecting controls (FIG. 6A). The ability to restore spine density in Shank3 knock down neurons by direct activation of Akt suggested that inhibition of CLK2 should have a similar outcome, in an Akt-dependent manner. To this end, organotypic slices were treated for 24 hours with the CLK2-inhibitor, TG003. As with SC79 treatments, TG003-mediated inhibition increased spine density in Shank3 knock down neurons to control levels (FIG. 6B). Critically, this effect was dependent on Akt activity as it was blocked by inclusion of 10 μM of an Akt inhibitor (Akti) in TG003 treatments (FIG. 6B). Pre-treatment with Akti (also called Akt inhibitor VIII) blocked BDNF-induced Akt phosphorylation in primary neurons (FIG. 5D). It was confirmed that Akt inhibition in wild type neurons is sufficient to reduce spine density and thereby phenocopy the effect of Shank3 deficiency on reducing spine density via downstream Akt attenuation (data unshown).

FIGS. 7A-7H show that knock-down of CLK2 restores dendritic spine density in Shank3-deficient neurons and Akt-activity inhibition was sufficient to reduce spine density. Neurons were infected with lentiviruses expressing either a shRNA specific for Shank3 or a control shRNA on DIV 2, and harvested for Western blotting on DIV 6, 9, 12, or 16. FIG. 7A shows the time course of Shank3 knockdown in primary neurons. FIG. 7B shows biolistically transfected hippocampal CA1 pyramidal neuron in organotypic slice culture. Dendritic spine quantification was on apical secondary dendrites (lower right). FIG. 7C and FIG. 7D show knockdown of Shank3 with additional shRNAs reduced dendritic spine density of hippocampal CA1 pyramidal neurons in organotypic slice cultures, which was corrected by 24 h pre-treatment with CLK2-inhibitor TG003. Neurons were fixed for staining on DIV 14. FIG. 7E shows that the reduced spine density in Shank3 knockdown neurons were rescued by re-expression of non-targeted GFP-Shank3. The shShank3-1 targets the 3′UTR of endogenous Shank3 mRNA and does not knockdown exogenously expressed GFP-Shank3.

FIG. 7F shows CLK2 shRNAs increased Akt-phosphorylation in primary neurons. Neurons were transduced with lentiviruses harboring five unique CLK2 shRNAs on DIV 6. The target sequences of the five CLK2 shRNAs are shown in Table 1. On DIV 16, half volume of neuron growth medium was removed and maintained at 37° C. Neurons were then treated with 20 μM TG003 15 min prior to 30 min BDNF stimulation (50 ng/ml), as indicated. Stimulation medium containing BDNF was then removed and replaced with unused growth medium for an additional 30 min. TG003, where indicated, was maintained throughout the experiment. Cell lysates were prepared in RIPA buffer prior to SDS-PAGE and Western blotting. As shown in FIG. 7F, CLK2 knock-down increased Akt-phosphorylation. A luciferase-targeting shRNA sequence was used as a control: AACTTACGCTGAGTACTTCGA (SEQ ID NO: 4). FIG. 7G shows knockdown of CLK2 by shRNA corrected spine density impairment caused by Shank3 deficiency. Co-transfection of GFP-expressing CLK2 shRNA #2 plasmid (shCLK2-2 as shown in FIG. 7F) with mCherry-expressing Shank3 shRNA plasmids corrected impaired dendritic spine density caused by Shank3 knock-down in hippocampal organotypic slice culture CA1 neurons. For shCont and shShank3 groups, total transfected DNA was normalized to that of shShank3+shCLK2-2 with shCont DNA. FIG. 7H shows that Akt-inhibition was sufficient to reduce dendritic spine density. Hippocampal organotypic slice cultures were biolistically transfected with Thy1-mGFP construct on DIV 1. Cultures were treated for 24 h with 10 μM Akti prior to fixation on DIV14.

TABLE 1
CLK2 shRNAs

| shRNA | Target Sequence | SEQ ID NO |
|---|---|---|
| shCLK2-1 | GCATCATCTTTGAGTACTACG | 5 |
| shCLK2-2 | CTTCTCGGATGATCAGAAAGA | 6 |
| shCLK2-3 | GAATATGTGGAATAGTGTAAA | 7 |
| shCLK2-4 | GAATAGTGTAAATATGACAGA | 8 |
| shCLK2-5 | ACATGTATATACTACTATTTA | 9 |

Given that SC79 and TG003 were able to recover spine numbers in Shank3 knock down neurons, the reinstatement of synaptic transmission was examined next. First, miniature excitatory postsynaptic currents (mEPSCs) were recorded from shRNA-expressing CA1 neurons in hippocampal organotypic slice cultures. Knock-down of Shank3 yielded a pronounced reduction of mEPSC frequency without impacting amplitude, an outcome that has previously been described in several Shank3 loss of function models (Peca et al., 2011, Nature 472: 437-442; Shcheglovitov et al., 2013, Nature 503: 267-271; Verpelli et al., 2011, The Journal of Biological Chemistry 286, 34839-34850) and by inhibition of insulin signaling to Akt (Lee et al., 2011, Neuropharmacology 61: 867-879). Similar to the effect on spine density, overnight treatment of Shank3 knock down neurons with 4 μg/ml SC79 completely restored mEPSC frequency to control levels (FIG. 6C). Second, Akt-activation and CLK2 inhibition were assayed for their impact on synaptic activity in PMDS neurons. Similar to an earlier report (Shcheglovitov et al., 2013, Nature 503, 267-271) and to Shank3 knock down in hippocampal slices (FIG. 6C), PMDS iPS-derived neurons exhibited a pronounced defect in the frequency of spontaneous EPSCs (sEPSCs) (FIG. 6D). sEPSCs were recorded from iPS-derived control or PMDS neurons at eight weeks in vitro. Strikingly, overnight treatment with SC79 or TG003, in an Akt-dependent fashion, again completely rescued this impairment (FIG. 6D). Importantly, the restorative effect of SC79 was also observed in neurons from a second, unrelated PMDS patient (PMDS-2; FIG. 6D).

Taken together, these results strongly suggest that Akt activity is impaired in Shank3 knock down or PMDS neurons and thereby insufficient for sustained neuronal function, specifically dendritic spine formation and synaptic transmission. As shown above, direct restoration of Akt activity, or by way of CLK2 inhibition, is sufficient to reverse these impairments Shank3 deficient or PMDS neurons.

### Example 5

### CLK2-Inhibition Rescues Deficits in Social Behavior Caused by Shank3 Deficiency

Generation of Shank3^{ΔC/ΔC} Mice

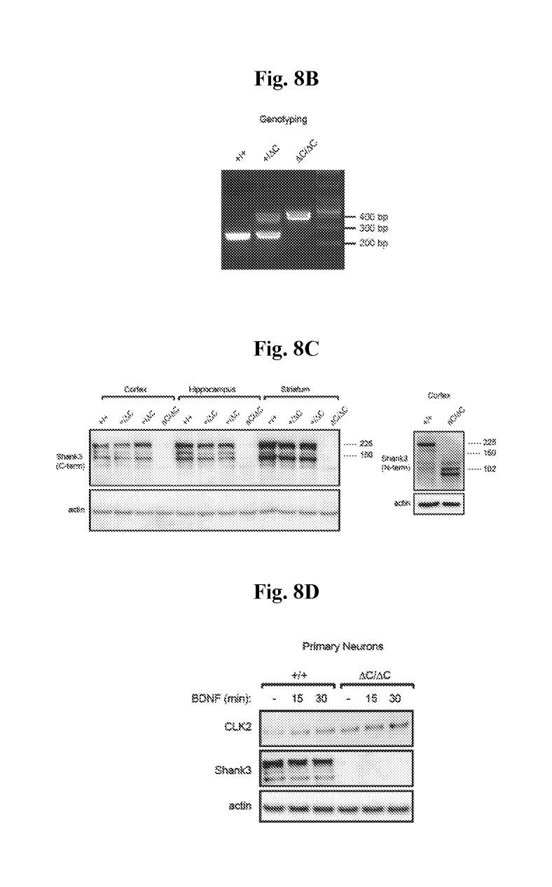

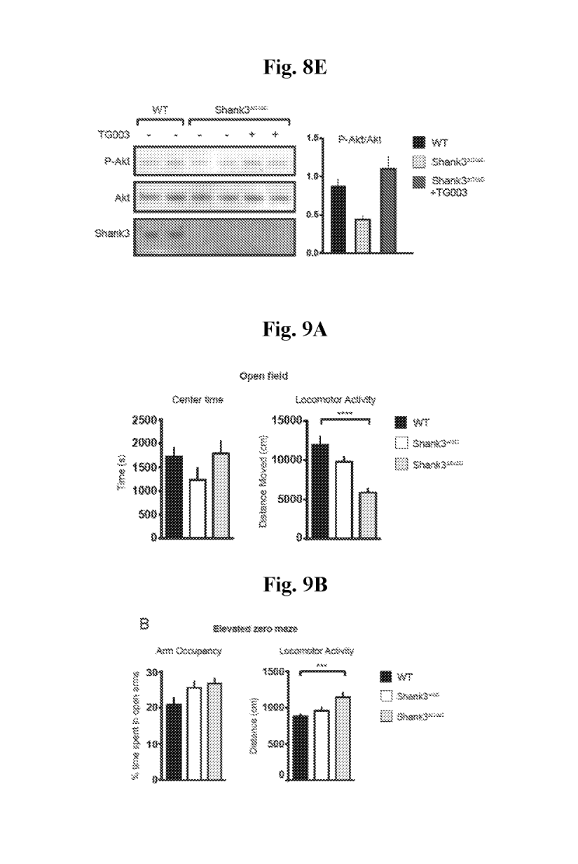

To determine whether the above findings extend to autism spectrum disorder (ASD)-like behaviors, a Shank3-deficient mouse model was generated by ablation of Shank3 exon 21 (FIGS. 8A and 8B) as previously described (Kouser et al., Journal of Neuroscience 33, 18448-18468, 2013; Duffney et al., Cell reports 11, 1400-1413, 2015). To generate Shank3 exon 21-deleted mice, the exon 21 genomic region of Shank3 (2464 bp in size) was replaced by homologous recombination with a loxP-TK_Neo-loxP cassette. The Neo cassette was flanked by a 3 kb 5′ homology arm and a 1.6 kb 3′ homology arm. Linearized targeting vector DNA was electroporated into C57BL6/J ES cells, and G418 resistant ES clones were first screened by nested PCR, and then subjected to Southern blot analysis. For Southern analysis, genomic DNA was digested with restriction enzyme(s), and hybridized with probes positioned outside the 5′ and 3′ homologous regions. Targeted ES clones were used for blastocyst injection, and chimeric males were mated with transgenic Cre-expressing C57B1/6 mice females to remove the neomycin resistance cassette. Animals without the neo cassette were used as F1 mice to establish the Shank3 exon 21-deleted (Shank3^{ΔC/ΔC}) colony.

The major Shank3 isoforms were absent in the homozygous mice (Shank3^{ΔC/ΔC}), while faster-migrating, truncated fragments were detected (FIG. 8C). Shank3^{ΔC/ΔC} neurons displayed excess CLK2 expression (FIG. 8D). In vivo treatment of Shank3^{ΔC/ΔC} mice with TG003 (30 mg/kg) increases Akt phosphorylation (FIG. 8E).

Behavioral Testing

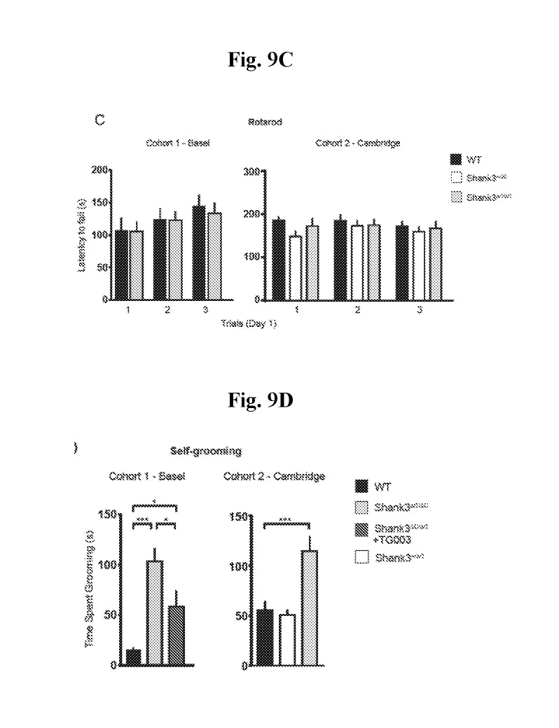

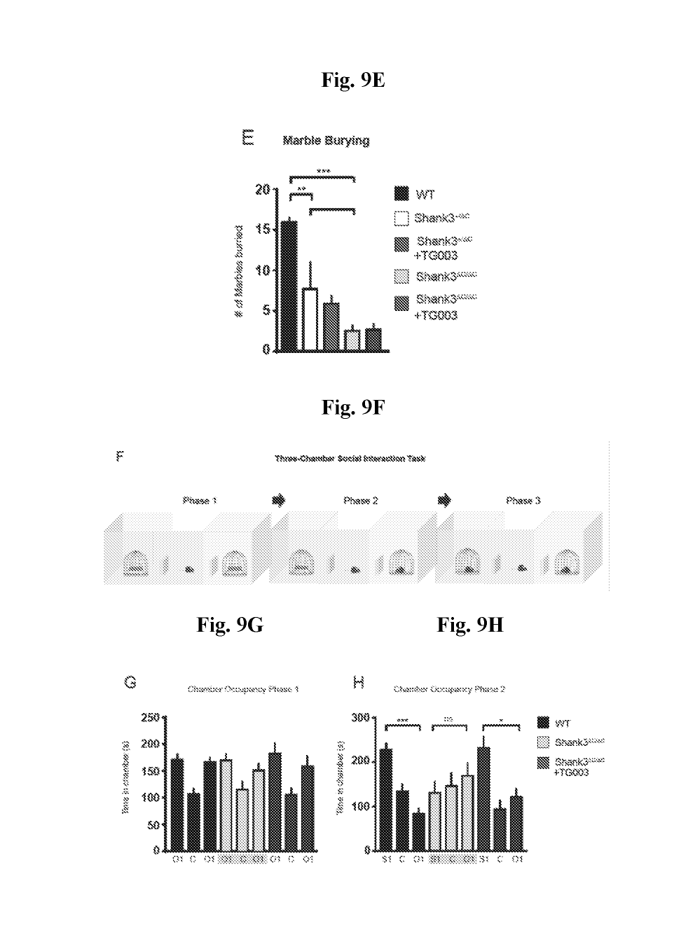

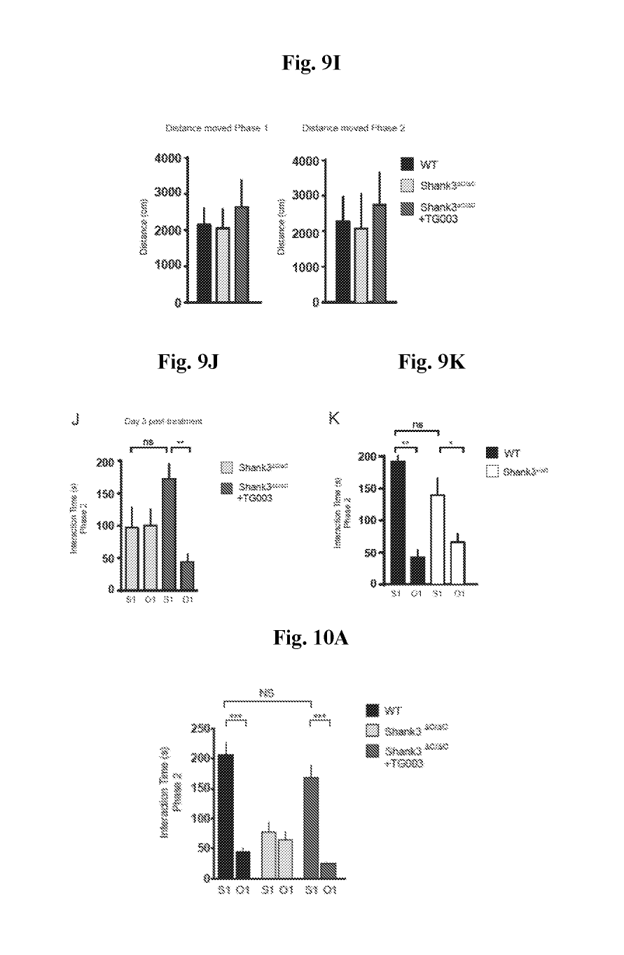

The three-chamber social interaction task was performed in a three-chambered arena with transparent walls and retractable doorways to allow mice access to flanking chambers (FIG. 9F). During testing, the arena was placed in a lighted, sound-proof box with no side-biasing features. A video camera was mounted above for recording. The test comprised three phases with different stimuli placed in the side chambers successively between phases. Each stimulus (social or inanimate) was placed within a small wire cage for immobilization while test mice were allowed to freely investigate the stimuli. In Phase 1, identical inanimate objects (inverted ceramic cups with blue stripe) were placed in both side chambers. An intruder mouse (same strain) was introduced in one side chamber as the social stimulus in Phase 2. In Phase 3, a second intruder mouse was placed in the other side chamber (novel social stimulus). Each phase consisted of a 7.5 minute exploration period with 5 minutes in the home cage between phases. The test animal was placed in the center chamber to start the task. Animals were habituated to the arena 24 hours before testing during which time they were allowed to freely explore the entire arena, with empty wire cages in side chambers, for 10 minutes. Social interaction time for a given phase was scored by cumulative social/investigative behaviors, in particular sniffing and actively seeking a stimulus. Proximity to a stimulus without investigation was not counted. Preference index was calculated by subtracting interaction time with one stimulus from that of the other stimulus in the same testing phase then dividing this by the sum of interaction times for both stimuli and converting this to a percentage. Positive scores indicate a preference for the first stimulus in the equation. Test mice were treated with 30 mg/kg TG003 or vehicle by intraperitoneal injection 6-8 hours before the start of testing.

To monitor spontaneous self-grooming, mice were placed in a clean cage (identical to the home cage), with minimal bedding to discourage digging, which was then placed in the sound-proof box for video monitoring. Animals were habituated in the new cage for 10 minutes before self-grooming was monitored during another 10 minutes. Cumulative grooming time was reported for this second 10 minute period.

To assess motor coordination on the rotarod device, mice were initially given a training session for 300 seconds at a constant rotation corresponding to the starting test speed. Mice were then tested over three trials in which the rotarod accelerated from either 5 to 50 revolutions per minute (Basel cohort) or from 4 to 40 revolutions per minute (Cambridge cohort) up to a maximum of 300 seconds per session or whenever the mouse falls from the rod.

To measure locomotor activity in the open-field, Omnitech Accuscan locomotor activity boxes measuring 40 cm×40 cm were used. Animals' locomotor performance was measured by beam breaks. Mice were acclimated to the testing room for a minimum of 30 minutes before testing, and then placed into the chambers for 120 minutes. The total distance traveled was measured. Assessment of anxiety was determined from the time spent in the center of the arena.

To measure anxiety, an elevated zero maze was used. This apparatus measures 52 cm wide by 50 cm high and is comprised of a ring walkway divided into four quadrants, two of which have open sides and two that are enclosed by high walls. The open arms are illuminated by high light levels (600-700 lux) which in pilot testing yields around 20% time spent in open arms in 8 week old C58B¹/6J mice. Mice are acclimated to the testing room for a minimum of 60 minutes before testing, and are then placed onto the maze for a 5 minute testing session. The % time spent on open arms and the total distance traveled are measured.

To assess avoidance behavior by marble burying, 20 black marbles were evenly distributed in rows in a novel home cage on top of 5 cm of fresh bedding and mice were left undisturbed for 30 minutes in the cage. The number of marbles to at least ⅔ depth were then recorded.

Results

FIGS. 9A-9K illustrate behavior characterization of the Shank3^{ΔC/ΔC} mice. Neither heterozygous (Shank3^{+/ΔC}) nor Shank3^{ΔC/ΔC} mice exhibited anxious behavior or locomotor skill impairments (FIGS. 9B & 9C). Both Shank3^{+/ΔC} and Shank3^{ΔC/ΔC} mice displayed avoidance behavior, assessed by marble burying, that was refractory to treatment with TG003 (FIG. 9E). In contrast, we observed that only Shank3^{ΔC/ΔC} mice exhibited excess self-grooming, a trait reflecting repetitive behaviors seen in ASD (FIG. 9D). Treatment of these mice with TG003 significantly decreased self-grooming although not to wild type frequency (FIG. 9D). Mice were then tested in a social motivation paradigm as shown in FIG. 8F. Wild type and Shank3^{+/ΔC} mice displayed a significant preference for social investigation, whereas Shank3^{ΔC/ΔC} mice did not (FIGS. 10A, 10B and 9H). In contrast, Shank3^{ΔC/ΔC} mice treated with TG003 recovered normal preference for social interaction (FIGS. 10A, 10B and 9). We observed that this effect persisted three days after treatment when a new cohort of animals was tested (FIG. 9J). The recovery of normal social behavior correlated with restored Akt phosphorylation in synaptosomal fractions (FIG. 8E). No significant preference was observed between groups when a second intruder was introduced (FIG. 10B). Thus, CLK2 inhibition rescues deficits in social behavior caused by Shank3 deficiency.

### Example 6

### IGF-1 Treatment Restores Dendritic Spine Density to Shank3 Knockdown Neurons, in an Akt-Dependent Manner

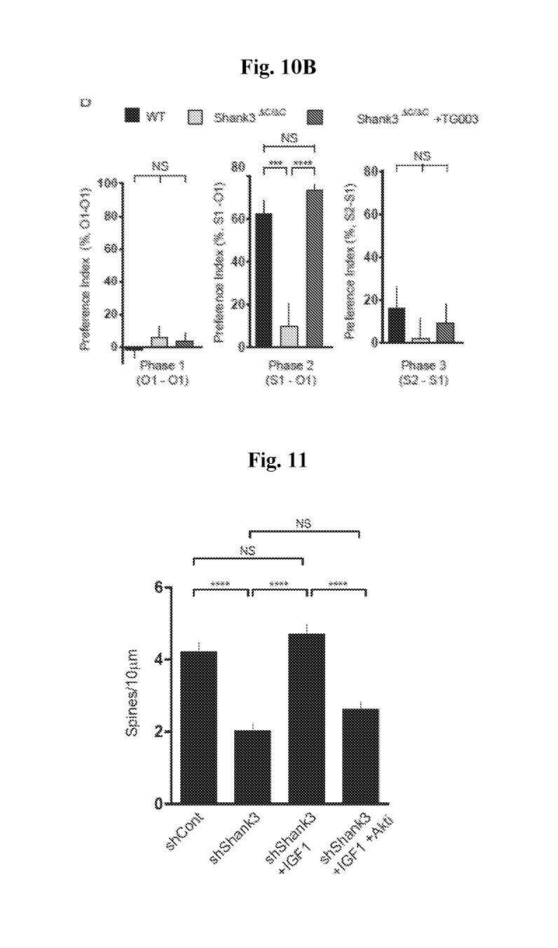

Cellular and behavioral impairments attributed to Shank3 loss of function have been corrected with IGF-1 treatment (Shcheglovitov et al., Nature 503, 267-271, 2013; Kolevzon et al., Molecular autism 5, 54, 2014; Bozdagi et al., Molecular autism 4, 9, 2013). Hippocampal organotypic slice culture was established as described in Example 4. Hippocampal organotypic slice culture neurons were transfected with shRNA vectors, and slices were treated for 24 h with 1 μg/ml IGF-1, or 1 μg/ml IGF-1 and 10 μM Akti, prior to fixation on DIV 14. IGF-1 treatment restored normal dendritic spine density to Shank3 knockdown neurons, in an Akt-dependent manner (FIG. 11). IGF-1 restores balance in signaling pathways likely by boosting Akt phosphorylation to counteract elevated dephosphorylation by PP2A. Thus, direct Akt-reactivation or CLK2 inhibition may be therapeutic targets for intervention in patients with PMDS.

Unless defined otherwise, the technical and scientific terms used herein have the same meaning as they usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. A method of treating Shank3 (SH3 and multiple ankyrin repeat domains 3) deficiency in a subject in need of treatment thereof, the method comprising:
administering a therapeutically effective amount of an agent that selectively decreases Cdc2-like kinase 2 (CLK2) protein level or kinase activity to the subject.

2. A method of treating Shank3 deficiency in a subject in need of treatment thereof, the method comprising:
administering a therapeutically effective amount of an agent that selectively increases protein kinase B (PKB or Akt) activity to the subject.

3. A method of treating Shank3 deficiency in a subject in need of treatment thereof, the method comprising:
administering a therapeutically effective amount of an agent that selectively decreases the activity of protein phosphatase 2 (PP2A) comprising B56β subunit (PP2A-B56β) to the subject.

4. The method of claim 1, wherein the method comprises the following steps:
assaying CLK2 protein level or kinase activity in a sample obtained from the subject;
determining that the subject's CLK2 protein level or kinase activity is higher than a reference CLK2 protein level or kinase activity; and
administering a therapeutically effective amount of an agent that selectively decreases CLK2 protein level or kinase activity to the subject.

5. A The method of claim 2, wherein the method comprises the following steps:
assaying Akt activity in a sample obtained from the subject;
determining that the subject's Akt activity is lower than a reference Akt activity; and
administering a therapeutically effective amount of an agent that selectively increases Akt activity to the subject.

6. The method of claim 3, wherein the method comprises the following steps:
assaying PP2A-B56β activity in a sample obtained from the subject;
determining that the subject's PP2A-B56β activity is higher than a reference PP2A-B56β activity; and
administering a therapeutically effective amount of an agent that selectively decreases the activity of PP2A-B56β to the subject.

7. A method of selecting a subject for treatment of Shank3 deficiency, the method comprising:
assaying CLK2 protein level or kinase activity in a sample obtained from a subject; and
selecting a subject whose CLK2 protein level or kinase activity is higher than a reference CLK2 level or kinase activity for the treatment of Shank3 deficiency.

8. A method of selecting a subject for treatment of Shank3 deficiency, the method comprising:
assaying the level of Akt activity in a sample obtained from the subject; and
selecting a subject whose Akt activity is lower than a reference Akt activity for the treatment of Shank3 deficiency.

9. A method of selecting a subject for treatment of Shank3 deficiency, the method comprising:
assaying PP2A-B56β activity in a sample obtained from the subject; and
selecting a subject whose PP2A-B56β activity is higher than a reference PP2A-B56β activity for the treatment of Shank3 deficiency.

10. A method of monitoring a treatment of Shank3 deficiency in a subject, the method comprising:
assaying the level of Akt activity in a first sample obtained from the subject before the treatment to obtain a first level of Akt activity;
assaying the level of Akt activity in a second sample obtained from the subject during or after the treatment to obtain a second level of Akt activity; and
comparing the first level with the second level.

11. The method of any of claim 1, wherein the Shank3 deficiency is selected from Phelan-McDermid syndrome, autism spectrum disorder, intellectual disability, or schizophrenia.

12. The method of claim 1, the method further comprising administering a second agent that treats Shank3 deficiency to the subject.

13. The method of claim 12, wherein the second agent is risperidone.

14. The method of claim 10, wherein the treatment of Shank3 deficiency is selected from an agent that selectively decreases CLK2 protein level or kinase activity, an agent that selectively decreases PP2A-B56β activity, or an agent that selectively increases Akt activity.

15. The method of claim 1, wherein the agent that selectively decreases CLK2 protein level or kinase activity is selected from a RNAi agent, an antisense oligonucleotide, a ribozyme, an aptamer, an antibody or derivative thereof, or a low molecular weight compound.

16. The method of claim 1, wherein the agent that selectively decreases CLK2 protein level or kinase activity is a low molecular weight compound.

17. The method of claim 1, wherein the agent that selectively decreases CLK2 protein level or kinase activity is TG003.

18. The method of claim 1, wherein the agent that selectively decreases CLK2 protein level or kinase activity is administered to the subject through an oral, intravenous, intracranial, or intranasal route.

19. The method of claim 2, wherein the agent that selectively increases Akt activity is a low molecular weight compound or an antibody or derivative thereof.

20. The method of claim 2, wherein the agent that selectively increases Akt activity is SC79.

21. The method of claim 2, wherein the agent that selectively increases Akt activity is selected from rapamycin, insulin-like growth factor-1 (IGF-1), insulin-like growth factor-2 (IGF-2), Brain-derived neurotrophic factor (BDNF), Epidermal growth factor (EGF), CC1-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, platelet derived growth factor, or granulocyte colony-stimulating factor (G-CSF).

22. The method of claim 2, wherein the agent that selectively increases Akt activity is administered to the subject through an oral, intravenous, intracranial, or intranasal route.

23. The method of claim 3, wherein the agent that selectively decreases PP2A-B56β activity is selected from a RNAi agent, an antisense oligonucleotide, a ribozyme, an aptamer, an antibody or derivative thereof, a low molecular weight compound, or a phosphorylation-deficient variant of B56β regulatory subunit.

24. The method of claim 3, wherein the agent that selectively decreases PP2A-B56β activity is a low molecular weight compound.

25. The method of claim 3, wherein the agent that selectively decreases PP2A-B56β activity is selected from okadaic acid, calyculin A, cantharidic acid, or cantharidin.

26. The method of claim 3, wherein the agent that selectively decreases PP2A-B56β activity is administered to the subject through an oral, intravenous, intracranial, or intranasal route.

27. The method of claim 4, wherein the reference CLK2 level or kinase activity is the level of CLK2 protein or kinase activity in a sample obtained from a healthy subject.

28. The method of claim 4, wherein CLK2 protein level or kinase activity in a sample is determined by an assay selected from a kinase assay, immunohistochemistry, Western blotting, immunofluorescent assay, radioimmunoassay (RIA), enzyme-linked immunosorbent assay (ELISA), or homogeneous time resolved fluorescence (HTRF).

29. The method of claim 5, wherein the reference Akt activity is the level of Akt activity in a sample obtained from a healthy subject.

30. The method of claim 5, wherein the level of Akt activity is determined by an assay selected from a kinase assay, immunohistochemistry, Western blotting, immunofluorescent assay, radioimmunoassay (RIA), enzyme-linked immunosorbent assay (ELISA), or homogeneous time resolved fluorescence (HTRF).

31. The method of claim 6, wherein the reference PP2A-B56β activity is the level of PP2A-B56β activity in a sample obtained from a healthy subject.

32. The method of claim 6, wherein the level of PP2A-B56β activity is determined by an assay selected from a phosphatase assay, immunohistochemistry, Western blotting, immunofluorescent assay, radioimmunoassay (RIA), enzyme-linked immunosorbent assay (ELISA), or homogeneous time resolved fluorescence (HTRF).

33. The method of claim 4, further comprising assaying the level or activity of a second protein in the sample.

34. The method of claim 4, wherein the sample is a cellular or tissue sample.

35. The method of claim 4, wherein the sample is a cellular or tissue sample comprising olfactory neurons obtained through nasal biopsy, induced pluripotent stem cell (iPS)-derived neurons, or cerebrospinal fluid.

36.-53. (canceled)
